SEQUENCE LISTING (101 sequences)

SEQ ID NO: 1 - DNA, 50 bp, synthetic construct
  source 1..50: mol_type = other DNA
catcaccatcaccatcacgacatgattttttataaaagcaaaagattagc

SEQ ID NO: 2 - DNA, 66 bp, synthetic construct
  source 1..66: mol_type = other DNA
ggatttgttggtatttggtcatatgtatatcctcctttatacaccttctttattgtgtttaaattc

SEQ ID NO: 3 - DNA, 22 bp, synthetic construct
  source 1..22: mol_type = other DNA
atgaccaaataccaacaaatcc

SEQ ID NO: 4 - DNA, 61 bp, synthetic construct
  source 1..61: mol_type = other DNA
gttattctttattgattgtcccatatgtatatcctcctctattttataaatcgcacatacc

SEQ ID NO: 5 - DNA, 24 bp, synthetic construct
  source 1..24: mol_type = other DNA
atgggacaatcaataaagaataac

SEQ ID NO: 6 - DNA, 44 bp, synthetic construct
  source 1..44: mol_type = other DNA
gagtccaagctcagctaattactaacgaatgatgctccaaaatg

SEQ ID NO: 7 - DNA, 56 bp, synthetic construct
  source 1..56: mol_type = other DNA
ggatcgcatcaccatcaccatcacggaatgattttttataaaagcaaaagattagc

SEQ ID NO: 8 - DNA, 46 bp, synthetic construct
  source 1..46: mol_type = other DNA
ggtattttcatatgtatatcctcctttatacaccttctttattgtg

SEQ ID NO: 9 - DNA, 37 bp, synthetic construct
  source 1..37: mol_type = other DNA
aggaggatatacatatgaaaatacctttttcaccacc

SEQ ID NO: 10 - DNA, 57 bp, synthetic construct
  source 1..57: mol_type = other DNA
caaatgtatatcctcctctagtggtggtggtggtggtgtcggttttctgcaattacg

SEQ ID NO: 11 - DNA, 43 bp, synthetic construct
  source 1..43: mol_type = other DNA
ccactagaggaggatatacatttgatccgtttttttgatatcg

SEQ ID NO: 12 - DNA, 44 bp, synthetic construct
  source 1..44: mol_type = other DNA
gagtccaagctcagctaattagagctcctaacgaatgatgctcc

SEQ ID NO: 13 - DNA, 54 bp, synthetic construct
  source 1..54: mol_type = other DNA
gcatcattcgttaggagctcaggaggatatacatatgaaattggctgtcatttc

SEQ ID NO: 14 - DNA, 70 bp, synthetic construct
  source 1..70: mol_type = other DNA
caagctcagctaattagagcttcacttgtcgtcatcgtctttgtagtcacgtactgttttatagattatg

SEQ ID NO: 15 - DNA, 54 bp, synthetic construct; identical to SEQ ID NO: 13
  source 1..54: mol_type = other DNA

SEQ ID NO: 16 - DNA, 25 bp, synthetic construct
  source 1..25: mol_type = other DNA
ccttcaacgtactgttttatagatt

SEQ ID NO: 17 - DNA, 62 bp, synthetic construct
  source 1..62: mol_type = other DNA
aatctataaaacagtacgttgaaggaggatatacatatgaggaagatattattaacatatgg

SEQ ID NO: 18 - DNA, 83 bp, synthetic construct
  source 1..83: mol_type = other DNA
caagctcagctaattagagcttcacttgtcgtcatcgtctttgtagtctgacatcataaatttattacatatattaattaatc

SEQ ID NO: 19 - DNA, 54 bp, synthetic construct; identical to SEQ ID NO: 13
  source 1..54: mol_type = other DNA

SEQ ID NO: 20 - DNA, 25 bp, synthetic construct
  source 1..25: mol_type = other DNA
ccttcaacgtactgttttatagatt

SEQ ID NO: 21 - DNA, 62 bp, synthetic construct; identical to SEQ ID NO: 17
  source 1..62: mol_type = other DNA

SEQ ID NO: 22 - DNA, 57 bp, synthetic construct
  source 1..57: mol_type = other DNA
ctcatatgtatatcctccttcatgacatcataaatttattacatatattaattaatc

SEQ ID NO: 23 - DNA, 36 bp, synthetic construct
  source 1..36: mol_type = other DNA
catgaaggaggatatacatatgagggatggaaagcc

SEQ ID NO: 24 - DNA, 78 bp, synthetic construct
  source 1..78: mol_type = other DNA
caagctcagctaattagagcttcacttgtcgtcatcgtctttgtagtctttatgtaaaagatttaaatatattctttc

SEQ ID NO: 25 - DNA, 54 bp, synthetic construct; identical to SEQ ID NO: 13
  source 1..54: mol_type = other DNA

SEQ ID NO: 26 - DNA, 25 bp, synthetic construct
  source 1..25: mol_type = other DNA
ccttcaacgtactgttttatagatt

SEQ ID NO: 27 - DNA, 62 bp, synthetic construct; identical to SEQ ID NO: 17
  source 1..62: mol_type = other DNA

SEQ ID NO: 28 - DNA, 57 bp, synthetic construct; identical to SEQ ID NO: 22
  source 1..57: mol_type = other DNA

SEQ ID NO: 29 - DNA, 36 bp, synthetic construct; identical to SEQ ID NO: 23
  source 1..36: mol_type = other DNA

SEQ ID NO: 30 - DNA, 42 bp, synthetic construct
  source 1..42: mol_type = other DNA
cctccttcatttatgtaaaagatttaaatatattctttcgag

SEQ ID NO: 31 - DNA, 61 bp, synthetic construct
  source 1..61: mol_type = other DNA
aaatcttttacataaatgaaggaggatatacatatgaaaaaaaaatatgactatctaattg

SEQ ID NO: 32 - DNA, 66 bp, synthetic construct
  source 1..66: mol_type = other DNA
caactacagcaaatatcttcatatgtatatcctccttcataagtcactatttataactttttccac

SEQ ID NO: 33 - DNA, 22 bp, synthetic construct
  source 1..22: mol_type = other DNA
atgaagatatttgctgtagttg

SEQ ID NO: 34 - DNA, 72 bp, synthetic construct
  source 1..72: mol_type = other DNA
caagctcagctaattagagctttacttgtcgtcatcgtctttgtagtcataggtagctccattttttttacg

SEQ ID NO: 35 - DNA, 54 bp, synthetic construct; identical to SEQ ID NO: 13
  source 1..54: mol_type = other DNA

SEQ ID NO: 36 - DNA, 25 bp, synthetic construct
  source 1..25: mol_type = other DNA
ccttcaacgtactgttttatagatt

SEQ ID NO: 37 - DNA, 62 bp, synthetic construct; identical to SEQ ID NO: 17
  source 1..62: mol_type = other DNA

SEQ ID NO: 38 - DNA, 57 bp, synthetic construct; identical to SEQ ID NO: 22
  source 1..57: mol_type = other DNA

SEQ ID NO: 39 - DNA, 36 bp, synthetic construct; identical to SEQ ID NO: 23
  source 1..36: mol_type = other DNA

SEQ ID NO: 40 - DNA, 28 bp, synthetic construct
  source 1..28: mol_type = other DNA
gtaaaagatttaaatatattctttcgag

SEQ ID NO: 41 - DNA, 72 bp, synthetic construct
  source 1..72: mol_type = other DNA
ctcgaaagaatatatttaaatcttttacataaatgaaggaggatatacatatgaccaaatacgaaaaaatcc

SEQ ID NO: 42 - DNA, 53 bp, synthetic construct
  source 1..53: mol_type = other DNA
caagctcagctaattagagctttattttttatcatttataaagcttatatacc

SEQ ID NO: 43 - DNA, 54 bp, synthetic construct; identical to SEQ ID NO: 13
  source 1..54: mol_type = other DNA

SEQ ID NO: 44 - DNA, 57 bp, synthetic construct; identical to SEQ ID NO: 22
  source 1..57: mol_type = other DNA

SEQ ID NO: 45 - DNA, 36 bp, synthetic construct; identical to SEQ ID NO: 23
  source 1..36: mol_type = other DNA

SEQ ID NO: 46 - DNA, 28 bp, synthetic construct
  source 1..28: mol_type = other DNA
gtaaaagatttaaatatattctttcgag

SEQ ID NO: 47 - DNA, 72 bp, synthetic construct; identical to SEQ ID NO: 41
  source 1..72: mol_type = other DNA

SEQ ID NO: 48 - DNA, 53 bp, synthetic construct; identical to SEQ ID NO: 42
  source 1..53: mol_type = other DNA

SEQ ID NO: 49 - DNA, 65 bp, synthetic construct
  source 1..65: mol_type = other DNA
gctttataaatgataaaaaataaaggaggatatacatatgaaaaaaaaatatgactatctaattg

SEQ ID NO: 50 - DNA, 66 bp, synthetic construct; identical to SEQ ID NO: 32
  source 1..66: mol_type = other DNA

SEQ ID NO: 51 - DNA, 22 bp, synthetic construct
  source 1..22: mol_type = other DNA
atgaagatatttgctgtagttg

SEQ ID NO: 52 - DNA, 48 bp, synthetic construct
  source 1..48: mol_type = other DNA
caagctcagctaattagagctttaataggtagctccattttttttacg

SEQ ID NO: 53 - DNA, 54 bp, synthetic construct; identical to SEQ ID NO: 13
  source 1..54: mol_type = other DNA

SEQ ID NO: 54 - DNA, 33 bp, synthetic construct
  source 1..33: mol_type = other DNA
cctcctttaataggtagctccattttttttacg

SEQ ID NO: 55 - DNA, 54 bp, synthetic construct
  source 1..54: mol_type = other DNA
tggagctacctattaaaggaggatatacatatgggacaatcaataaagaataac

SEQ ID NO: 56 - DNA, 37 bp, synthetic construct
  source 1..37: mol_type = other DNA
cctccttcattgtttaaatattgaaagtaataatttc

SEQ ID NO: 57 - DNA, 61 bp, synthetic construct
  source 1..61: mol_type = other DNA
caatatttaaacaatgaaggaggatatacatatgactagtacttctttctttattattaag

SEQ ID NO: 58 - DNA, 45 bp, synthetic construct
  source 1..45: mol_type = other DNA
aagctcagctaattagagctttaaaatttgattttggcattaaac

SEQ ID NO: 59 - DNA, 54 bp, synthetic construct; identical to SEQ ID NO: 13
  source 1..54: mol_type = other DNA

SEQ ID NO: 60 - DNA, 33 bp, synthetic construct; identical to SEQ ID NO: 54
  source 1..33: mol_type = other DNA

SEQ ID NO: 61 - DNA, 54 bp, synthetic construct; identical to SEQ ID NO: 55
  source 1..54: mol_type = other DNA

SEQ ID NO: 62 - DNA, 52 bp, synthetic construct
  source 1..52: mol_type = other DNA
caagctcagctaattagagcttcattgtttaaatattgaaagtaataatttc

SEQ ID NO: 63 - DNA, 54 bp, synthetic construct; identical to SEQ ID NO: 13
  source 1..54: mol_type = other DNA

SEQ ID NO: 64 - DNA, 33 bp, synthetic construct; identical to SEQ ID NO: 54
  source 1..33: mol_type = other DNA

SEQ ID NO: 65 - DNA, 54 bp, synthetic construct; identical to SEQ ID NO: 55
  source 1..54: mol_type = other DNA

SEQ ID NO: 66 - DNA, 52 bp, synthetic construct; identical to SEQ ID NO: 62
  source 1..52: mol_type = other DNA

SEQ ID NO: 67 - DNA, 71 bp, synthetic construct
  source 1..71: mol_type = other DNA
gaaaggctctttacgttagatgagcttatcagattaaaattaattgcatgacattacacgtcttgagcgat

SEQ ID NO: 68 - DNA, 71 bp, synthetic construct
  source 1..71: mol_type = other DNA
gcacaaacggcaccaaacaaaccagaaccaacaatgatataatcgtacatatgaatatcctccttagttcc

SEQ ID NO: 69 - DNA, 70 bp, synthetic construct
  source 1..70: mol_type = other DNA
tcaacagtcaagcagttttggaaaagttatcatcattataaaggtaaaactgaatatcctccttagttcc

SEQ ID NO: 70 - DNA, 67 bp, synthetic construct
  source 1..67: mol_type = other DNA
ttgtatagataagaagtgagttttaactcacttcttaaacttgtttattcattgtgtaggctggagc

SEQ ID NO: 71 - DNA, 18 bp, synthetic construct
  source 1..18: mol_type = other DNA
tggctgctattgggcgaa

SEQ ID NO: 72 - DNA, 22 bp, synthetic construct
  source 1..22: mol_type = other DNA
tccaccgatatgatttcttttc

SEQ ID NO: 73 - DNA, 16 bp, synthetic construct
  source 1..16: mol_type = other DNA
tatcccaatggcatcg

SEQ ID NO: 74 - DNA, 19 bp, synthetic construct
  source 1..19: mol_type = other DNA
accctaattcacgtactcc

SEQ ID NO: 75 - DNA, 1773 bp, Campylobacter jejuni
  source 1..1773: mol_type = genomic DNA
atgattttttataaaagcaaaagattagcattttttttaatttcagatattgttttaattttacttagcgtttatctggctttttctttgagatttagtggagatattccgagcattttttatcatggcatgatggtttctgctattattttgcttgttttaaaactttcatttttatttgtttttagaatttacaaagtagcttggagatttttttctctcaatgaagcaagaaaaatttttatcgctttgcttttagctgagttttgtttttttcttattttttatttttttagtgatttttttaatccttttccaagaagtgctattgtgatagattttgttctttcttatatgtttataggtactttaagaattagcaaaagaatgcttgtggattttaaaccttctagaatgaaagaagaagaaactccttgtattgtagtaggggcaacttctaaggctttgcatttgttaaaaggtgcaaaagaaggttctttagggctttttcctgtaggcgtagttgatgcgagaaaagagcttatagggacttattgtgataaatttattgtagaagaaaaagaaaaaataaaatcttatgcagaacaaggggtaaaaactgccattattgctttaagacttgaacaagaagagcttaaaaaactttttgaagaacttgtagcttatggtatttgcgatgtaaaaatattttcttttacaagaaacgaagcaagagatatcagtatagaagacttgcttgctagaaaaccaaaagatttagatgatagtgctgtggcggcttttttaaaagataaggtagttttggtaagtggagcaggtggaactataggcagtgaactttgtaagcaatgtattaaatttggtgctaagcatcttatcatggttgatcatagtgagtataatctttataagatcaatgatgatttaaatttatataaagaaaaaattactcctattttactgagtattttagataagcaaagtttagatgaggtattaaaaacttataaacccgagcttattttacatgcagccgcttataaacatgtgcctctttgcgaacaaaatccacattcagcagtaatcaataatattttaggaactaaaattttatgcgatagtgctaaagaaaacaaagtagctaaatttgtgatgataagtacagataaagcagtacgaccaacaaatattatgggttgcactaagagagtttgcgagctttatactttaagtatgagtgatgaaaattttgaagttgcttgtgtgcgttttggtaatgttttaggttctagtggtagtgtgataccgaaatttaaagcacaaattgccaataatgagcctttaactttaacgcaccctgatatagtgcgttattttatgcttgtggctgaggcagtgcaacttgttttacaagctggagctatcgcaaaagggggagaactttttgttttggatatgggtaagcctgtgaaaatcatagatttagctaaaaaaatgcttttactttctaatcgcaatgatttagaaattaaaatcacaggcttaagaaaaggtgagaagctttatgaagagcttttgattgatgaaaatgatgctaaaacacaatatgagagtatttttgtagcaaagaatgagaaggttgatcttaattggcttaataaagagatagaaaatttacaaatatgtgaagatatttcagaggctttattaaagattgtacctgaatttaaacacaataaagaaggtgtataa

SEQ ID NO: 76 - protein, 590 aa, Campylobacter jejuni
MIFYKSKRLAFFLISDIVLILLSVYLAFSLRFSGDIPSIFYHGMMVSAIILLVLKLSFLFVFRIYKVAWRFFSLNEARKIFIALLLAEFCFFLIFYFFSDFFNPFPRSAIVIDFVLSYMFIGTLRISKRMLVDFKPSRMKEEETPCIVVGATSKALHLLKGAKEGSLGLFPVGVVDARKELIGTYCDKFIVEEKEKIKSYAEQGVKTAIIALRLEQEELKKLFEELVAYGICDVKIFSFTRNEARDISIEDLLARKPKDLDDSAVAAFLKDKVVLVSGAGGTIGSELCKQCIKFGAKHLIMVDHSEYNLYKINDDLNLYKEKITPILLSILDKQSLDEVLKTYKPELILHAAAYKHVPLCEQNPHSAVINNILGTKILCDSAKENKVAKFVMISTDKAVRPTNIMGCTKRVCELYTLSMSDENFEVACVRFGNVLGSSGSVIPKFKAQIANNEPLTLTHPDIVRYFMLVAEAVQLVLQAGAIAKGGELFVLDMGKPVKIIDLAKKMLLLSNRNDLEIKITGLRKGEKLYEELLIDENDAKTQYESIFVAKNEKVDLNWLNKEIENLQICEDISEALLKIVPEFKHNKEGV

SEQ ID NO: 77 - DNA, 1032 bp, Vibrio vulnificus
  source 1..1032: mol_type = genomic DNA
atgaccaaatacgaaaaaatccaacaagatttagttgaatcaccaaaaacatggttgataaccggtgttgctggttttattggctccaatttactagaaaaactactaaagcttgatcaaaccgtggtggggttagataactttgccacgggtcatcaacataacttagatgaagttaaaagtttagtaacagaagcccaatggcaacgatttacctttattgaaggcgatatacgtaatcctgagacctgtgtagcagcggttcaaggtgtagactatgtattacaccaagcggcactagggtctgtgccccgttcaattaatgatcctattactacaaattccgctaacatcactggttttttaaacatgttagtagcagccaaagatcagcaagtgaaaagctttacttatgccgcctcaagttcgacttatggtgatcaccctgcattgccgaaggtagaagaaaatattggcaacccgttatccccctacgccgtaaccaaatacgtaaatgaactttatgcgaatgtatttgcgcggacttatggttttaaaagtattggactgcgttattttaatgtgtttggaaaacgccaagatccaaatggtgcctatgcggctgttattccaaaatggacagcagcaatgattcaaggtgatgttatttatattaatggtgacggcgagactagtcgcgatttctgttatatagctaatacagtgcaaatgaatattcttgccgccaccgcaccagaagaagcaaaagacaagatttacaatgttgcagtaggagacagaacaaccttaaacgatctattctctgccattaaattggcattatcgaataatactgttgttgtttctcaagaacctatttatagagagtttcgtactggtgatgtaaggcattctcaggctgatgttaataaagctaagcagtatttaggctatgcgccgtcgcaccgtattctagatggaatcaacgaagcaatgccatggtatataagctttataaatgataaaaaataa

SEQ ID NO: 78 - protein, 343 aa, Vibrio vulnificus
MTKYEKIQQDLVESPKTWLITGVAGFIGSNLLEKLLKLDQTVVGLDNFATGHQHNLDEVKSLVTEAQWQRFTFIEGDIRNPETCVAAVQGVDYVLHQAALGSVPRSINDPITTNSANITGFLNMLVAAKDQQVKSFTYAASSSTYGDHPALPKVEENIGNPLSPYAVTKYVNELYANVFARTYGFKSIGLRYFNVFGKRQDPNGAYAAVIPKWTAAMIQGDVIYINGDGETSRDFCYIANTVQMNILAATAPEEAKDKIYNVAVGDRTTLNDLFSAIKLALSNNTVVVSQEPIYREFRTGDVRHSQADVNKAKQYLGYAPSHRILDGINEAMPWYISFINDKK

SEQ ID NO: 79 - DNA, 223 bp, Bacteroides fragilis
  source 1..223: mol_type = genomic DNA
atgaaaatacctttttcaccaccttatattgacgaggcggtcatcaacgaagtcgttgattcgttacgttccggttggatcacatccggtccgaaagtgaaggctttggaggaagaaattaaatctttctcaggagcaaaagaagtgctttgtgtcaattcctggacatcaggagctattatgatgttgcgttggctgggagtgaaggagggcgatgaagtga

SEQ ID NO: 80 - protein, 407 aa, Bacteroides fragilis
MKIPFSPPYIDEAVINEVVDSLRSGWITSGPKVKALEEEIKSFSGAKEVLCVNSWTSGAIMMLRWLGVKEGDEVIVPAYTYSATALAVLHAGAKPVMVDSGTDFNISVEAVRKAITPKTKAIIPVDIAGFPCDYERIMALVQEPEMVKLFRSESPVQEKLGRILVMNDAAHSLGARYSSRQRTGCETDVAIFSLHAVKNVTTAEGGAICLNLPKPFDNTELYKELRMTSLNCQTKDAFSKSKAGGWRYDIVGFGMKINMADVNAAIGLAQIREYPELLKERKRVFNAYSDAFSACDWAIVPPSVDGEKESSYHIYALRIKDFTEEQRDRMIDEIAKSEVAVNVHFIPMPMLSFFKSMGYDIKDYPQAYQNFKSEISLPIYPQLDSEKLNFIIETVKAAYATVIAENR

SEQ ID NO: 81 - DNA, 597 bp, Bacteroides fragilis
  source 1..597: mol_type = genomic DNA
ataagttgcttgatccgtttttttgatatcgttttttcccttttgggaattcttctgctttctcccgtttttcttcttctttatatagccatttgtctcgaaagtaaaggaggtggtttctataaacagctccgtgtgggaaggtacggaggggatttttatgtttataaatttcgttcgatgcgtgtaggggctgataaaaaggggttgataacggtaggagggagagatccgagaataactcgtaccggatacctgatccggaagtataaattggatgaattgcctcagttgtttaacgtactgaaaggagatatgagtctggtagggccccgtcctgaggttcgtaagtatgtggatttatataccgatgaacaaaagaaagtattgagtgtacgtccggggattacggattatgcttctattgagtatgtagatgaaaatatgattcttggagaggcttcggatcctgacagagcatatatagaacagattatgcccgataagatacggtataatatgaaatatatctgtaatcgttcggtgaaggaatattttaaaattatatttctgacattttggagcatcattcgttag

SEQ ID NO: 82 - protein, 198 aa, Bacteroides fragilis
MSCLIRFFDIVFSLLGILLLSPVFLLLYIAICLESKGGGFYKQLRVGRYGGDFYVYKFRSMRVGADKKGLITVGGRDPRITRTGYLIRKYKLDELPQLFNVLKGDMSLVGPRPEVRKYVDLYTDEQKKVLSVRPGITDYASIEYVDENMILGEASDPDRAYIEQIMPDKIRYNMKYICNRSVKEYFKIIFLTFWSIIR

SEQ ID NO: 83 - DNA, 807 bp, Bacteroides fragilis
  source 1..807: mol_type = genomic DNA
atgaaattggctgtcatttcttctatctacaaaaatgattcgctttctaaaataaagcaatccttacaaagtttgttgaatcaagaaaatgttacttgtgatatttatttatattgtgatggtcctgtttcgattgatattgtgcatttcttagactcatttgatcaaaactcactttatatatttaagcaagaacaaaatagaggattggctcatgcactaaatgaattattgaatatcgttcttacaaaagaatacgaatatattgctcgtatggatgctgatgatatttccatgccggaacgttttgtgaagcagatcgcatttatgaattcacatccggatattgattgccttggtacttgggcaatagaaattgatgatgacggaaaagaatattttcgaaaaaaaatgccgataactcatgaagaatgtttggaactgtttaaaaagcgggattgtatgattcatcctactgtgatgtttcgacgttcatattttgagaaagctggtttatatcccgaagacacctactttggagaagatactatgatgtgggctaaaggttttaaaagtggatgtaaatttgccaatgtacccgaatatctgtttaagtttagattggattcaaacttctttgaaagaaggcgggggtggaaacatgcaaaatctatttatactttacgtcatcgtgttaatagaatgttagggttcggatggaaggaagattgttatgcattgctatatgctatggccaaattaatgcctaaatctattcttgacataatctataaaacagtacgttga

SEQ ID NO: 84 - protein, 268 aa, Bacteroides fragilis
MKLAVISSIYKNDSLSKIKQSLQSLLNQENVTCDIYLYCDGPVSIDIVHFLDSFDQNSLYIFKQEQNRGLAHALNELLNIVLTKEYEYIARMDADDISMPERFVKQIAFMNSHPDIDCLGTWAIEIDDDGKEYFRKKMPITHEECLELFKKRDCMIHPTVMFRRSYFEKAGLYPEDTYFGEDTMMWAKGFKSGCKFANVPEYLFKFRLDSNFFERRRGWKHAKSIYTLRHRVNRMLGFGWKEDCYALLYAMAKLMPKSILDIIYKTVR

SEQ ID NO: 85 - DNA, 1044 bp, Bacteroides fragilis
  source 1..1044: mol_type = genomic DNA
atgaggaagatattattaacatatggagatattaaaaccataaatattggtgattatattcagagtatcgccgctaaacaattttttgatgatgataattatatatttttcaatcgtgatgaattaaagttgtataaaggggaacctgcaaaagtcataatgaatgcttggatgacttataaaccatataattggcctccttcatctcaggtttatccattatttgttgcattacatatcaattcttctgcagaaagtcgctttttgtcacatgatagtattaagtatttaaaaaagtatgaacccatagggtgccgcgattatcatacaatgaatattttaaaaggaaaaggtgttaatgcttatttttcgggatgtcttactactactttgggtaaaacttataaatataatggaaaacgtgaggggatatacattgttgatccactttcatacatgcctaatgggaataatttttttgaaataatgaaagctgttgtacaaactgttttttatatgaagcctgtgctaaagattcttagaaattataaaaagaataatagatttactattaatatatccaaagtaggaataggacgtttattgttaataaccaagtcttatttgcttttgagaaagctagtagttcctgacgtattatacaatgcaatatttataacacaatttaatatgtctaatgaatattcttctgaatcagaaagatttgctcgggctgatgaattattaactaaaatggcctcagctcaatatgtgataacttctagaatccactgtgctcttccttgcttaggctttgagacccctgtggtttatattagaaatctatcagaaagcaagaagagcacttgtcgattagggggacttgaatctctatttaatgtgataactgtgaagggggaaaatgttacttccaatttttttgatggactatttaaacgagattcttcttttaagaataaaattgattttgtagaatatagagatcgattaattaatatatgtaataaatttatgatgtcatga

SEQ ID NO: 86 - protein, 347 aa, Bacteroides fragilis
MRKILLTYGDIKTINIGDYIQSIAAKQFFDDDNYIFFNRDELKLYKGEPAKVIMNAWMTYKPYNWPPSSQVYPLFVALHINSSAESRFLSHDSIKYLKKYEPIGCRDYHTMNILKGKGVNAYFSGCLTTTLGKTYKYNGKREGIYIVDPLSYMPNGNNFFEIMKAVVQTVFYMKPVLKILRNYKKNNRFTINISKVGIGRLLLITKSYLLLRKLVVPDVLYNAIFITQFNMSNEYSSESERFARADELLTKMASAQYVITSRIHCALPCLGFETPVVYIRNLSESKKSTCRLGGLESLFNVITVKGENVTSNFFDGLFKRDSSFKNKIDFVEYRDRLINICNKFMMS

SEQ ID NO: 87 - DNA, 1137 bp, Bacteroides fragilis
  source 1..1137: mol_type = genomic DNA
atgagggatggaaagcccattgaaatactgcatattgtagggactagacctgttggaggaattggtgctttattgaaaaatataaacacttctattgatttaaataaattccattttacatatgtgttttctgccgatagtaatattggagattttgataattatgtgagaaaattaggtagcgatattgttgtattcccttcatatcatttgaaatatttatttttatatttaaaggtgattttctgtttttataagagaaatgcaaaaaaatatgatataattcatgtacatagtgctaatacaggagtattagaccttttatttgcaaagatatatggaatacggataaggattttacatagtcacagtacaaaatattctagtaaaaaaataaggtccattagaaattattttttgcaatttcctacaatatatttagcaaacacttattttgcatgtggatacaaagctgctgaattcctttttggaaaaaagagacttgaaaaagtttatattattcataatgcaattttttctaaaaaatttatttataatgcaactgttagagagagagttagagttgagttgaaaatagagaatgactgtttactgttggggcatatcggtaactttacgaaagaaaagaatcatggttttttaatagatatattggaagaattattgaatattaatgacaatgttaagttgctgttagtaggggatggccaacttagaagtgaaatagaggaaaaagtaaaattaaaaagattgcagaagtatgtgtgctttttaggtcggagaactgatatatcagaattattacaagctatggattttttaatttttccttctttttttgaaggtctaccagtgactctgattgaagcgcaagcttctggtttaagatgtgtcgtttcggatcgtattactttggaaacaaagataacgaataatataagttatcttaatctcaatcgagatccttcaacttgggctgaagttgtttggaagttgtttgaagataaaacttttatcaggaaagatacgtctaaagaaatatgtgaatcaggatatgatatacatttagagagtgaatatctcgaaagaatatatttaaatcttttacataaatga

SEQ ID NO: 88 - protein, 378 aa, Bacteroides fragilis
MRDGKPIEILHIVGTRPVGGIGALLKNINTSIDLNKFHFTYVFSADSNIGDFDNYVRKLGSDIVVFPSYHLKYLFLYLKVIFCFYKRNAKKYDIIHVHSANTGVLDLLFAKIYGIRIRILHSHSTKYSSKKIRSIRNYFLQFPTIYLANTYFACGYKAAEFLFGKKRLEKVYIIHNAIFSKKFIYNATVRERVRVELKIENDCLLLGHIGNFTKEKNHGFLIDILEELLNINDNVKLLLVGDGQLRSEIEEKVKLKRLQKYVCFLGRRTDISELLQAMDFLIFPSFFEGLPVTLIEAQASGLRCVVSDRITLETKITNNISYLNLNRDPSTWAEVVWKLFEDKTFIRKDTSKEICESGYDIHLESEYLERIYLNLLHK

SEQ ID NO: 89 - DNA, 1095 bp, Bacteroides fragilis
  source 1..1095: mol_type = genomic DNA
atgaaaaaaaaatatgactatctaattgtcggagccggactttatggttctgtttttgcatataaagcgcaacggaatgggaaaaaatgtttgcttattgacaaacgtccacacaatggaggaaatatttattgcgaaaatattgagggtataaatgtacacaaatatggtgcacatatttttcatacttctaataaagaggtttgggactttgtaaatgccatagtggaatttaaccgctatactaattcccctattgccaattataaaggcaaattatataatttaccttttaatatgaatactttttatgctttatgggggacaaaaactccagaggaagcaaaaatgaagatagaagagcaaaggagagaagcggggattcttacgccgaagaatctagaagaacaggctatctctttagtaggaaaggatatttatgaaatattgataaaaggatatactgagaaacagtgggggcggaaagcaactgaacttcctgcttttataatcaagcgtttacctgtcagatttacttttgataataattactttaatgataaatatcaggggattcctgttggtggctataataaattgataaatggtttattagatggtattgaagttagaacaaatacagacttttttcaaaataaagaatattttaaaggtcttgcggataaaattctatttactggcaaaattgatgaatattataattatcaatttgggagattggaatatcgtactgttgaatttgaaacacaagtgcttgattgtgagaattatcaagggaatgcagttgtgaattatactgaaagggaagtgccttatactcgtattatagagcataaacattttgaatttggaacacagcccaaaaccgtaatttccaaagaatattcttcggaatggaaagatggtagtgaacccttttatcctgttaatgatgagaggaataattctttgtatttggagtataagaaattggtcgatagagagaagaatgttttgtttggagggagattagctgagtataagtattatgatatgaatgtgattgtggaaaaagttataaatagtgacttatga

SEQ ID NO: 90 - protein, 248 aa, Bacteroides fragilis
MKKKYDYLIVGAGLYGSVFAYKAQRNGKKCLLIDKRPHNGGNIYRKATELPAFIIKRLPVRFTFDNNYFNDKYQGIPVGGYNKLINGLLDGIEVRTNTDFFQNKEYFKGLADKILFTGKIDEYYNYQFGRLEYRTVEFETQVLDCENYQGNAVVNYTEREVPYTRIIEHKHFEFGTQPKTVISKEYSSEWKDGSEPFYPVNDERNNSLYLEYKKLVDREKNVLFGGRLAEYKYYDMNVIVEKVINSDL

SEQ ID NO: 91 - DNA, 876 bp, Bacteroides fragilis
  source 1..876: mol_type = genomic DNA
atgaagatatttgctgtagttgtgacctataatcgtttagcattactaaaaaaggtcatagatttacttaaaagacagacacgtcaattagatagtatagttatagtaaataatggctctactgatggaacaaaagaatggttagaacaggagaaaaatataacattaataaatcaatctaattctggtggggctggtggattcgaaacgggggtaaaatatgcatatgagaatgaggcagattggatttggatgatggatgatgatgtttttcctaatatagattgtttggaaaaactcttagggtggacgagtatttctcaatgtatccaaccacgtaggtattattcggatgatgtagaagtaaattttgaacaatggttggatccaattacttatagtaaatttggttattggcaagaaaaatcatttaataatggtaaaaagttttgtgctattaatgtaggctgttttgaagggatgtttgtttcgaaaagcattgttaataaaataggatttcctgataagcgtttttttatagcggaagatgatacaatatatgggtttgcagctagtttttataccaacgtaattttagtatctgatgctatcatggttcgtgctagaagatcatcggatcgtagcgttagcccaatgtatacttattatgcatgtcggaattttcatttagtatatgagtcacttaatttattattggataagaaaaataggtttttatatattaaatatatttatcaatttgttcatcagatttatttatctatttttttatatgataataagatgaaacacttaatatctgtttttcgtggcttttatgattgctttcgtaaaaaaaatggagctacctattaa

SEQ ID NO: 92 - protein, 291 aa, Bacteroides fragilis
MKIFAVVVTYNRLALLKKVIDLLKRQTRQLDSIVIVNNGSTDGTKEWLEQEKNITLINQSNSGGAGGFETGVKYAYENEADWIWMMDDDVFPNIDCLEKLLGWTSISQCIQPRRYYSDDVEVNFEQWLDPITYSKFGYWQEKSFNNGKKFCAINVGCFEGMFVSKSIVNKIGFPDKRFFIAEDDTIYGFAASFYTNVILVSDAIMVRARRSSDRSVSPMYTYYACRNFHLVYESLNLLLDKKNRFLYIKYIYQFVHQIYLSIFLYDNKMKHLISVFRGFYDCFRKKNGATY

SEQ ID NO: 93 - DNA, 1140 bp, Escherichia coli
  source 1..1140: mol_type = genomic DNA
atgatgaaatccaaaatgaaattgatgccattattggtgtcagtaaccttgataagcggttgcacagtacttccgggcagcaatatgtcgacgatgggcaaagacgtcatcaaacagcaggacgctgatttcgatctcgacaaaatggtgaatgtttatccgctgaccccgcgcctgattgaccaattacgcccacgcccgaatgtagcgcgccccaatatgacgctggaaagtgagatcgcgaattaccagtatcgcgtcgggccgggggacgttcttaatgtcaccgtctgggatcacccggaactcaccacgccagccggtcagtaccgcagctccagcgacaccggcaactgggtacagcctgacggcactatgttttacccgtatatcggcaaggtccacgtagtcgggaaaacgctcgctgaaatccgcagtgatattaccgggcgcttagcgacgtacatcgctgacccgcaggtggacgttaatatcgccgccttccgctcacaaaaggcctatatctccggtcaggtgaataaatccggtcaacaggcgatcaccaacgtgccactgactattctcgacgccatcaacgccgcaggtggcctgaccgacaccgctgactggcgcaacgtggtgctaacacacaatggtcgtgaagagcgcatttctttgcaggcgctgatgcaaaacggcgacctcaaccagaatcgcctgctttaccccggcgatattctctacgtgccacgtaatgatgatctgaaagtatttgtgatgggtgaagtgaagaaacagagcaccctgaaaatggactttagcggcatgaccctgactgaagccctgggcaatgctgaaggcatcgacatgaccacctccaacgccagcggcatctttgtcattcgtccgctgaaaggcgagggcgggcgtaacggcaagattgccaatatctaccagctggatatgtccgatgccacgtcgctggtgatggcgacagaattccgcctgcaaccttatgacgtggtgtatgtcaccaccgccccggtttcccgctggaaccgtctgatcaatcagttgctgccaactattagcggtgtccgttacatgacggatacagccagcgacattcataactggtaa

SEQ ID NO: 94 - protein, 379 aa, Escherichia coli
MMKSKMKLMPLLVSVTLISGCTVLPGSNMSTMGKDVIKQQDADFDLDKMVNVYPLTPRLIDQLRPRPNVARPNMTLESEIANYQYRVGPGDVLNVTVWDHPELTTPAGQYRSSSDTGNWVQPDGTMFYPYIGKVHVVGKTLAEIRSDITGRLATYIADPQVDVNIAAFRSQKAYISGQVNKSGQQAITNVPLTILDAINAAGGLTDTADWRNVVLTHNGREERISLQALMQNGDLNQNRLLYPGDILYVPRNDDLKVFVMGEVKKQSTLKMDFSGMTLTEALGNAEGIDMTTSNASGIFVIRPLKGEGGRNGKIANIYQLDMSDATSLVMATEFRLQPYDVVYVTTAPVSRWNRLINQLLPTISGVRYMTDTASDIHNW

SEQ ID NO: 95 - DNA, 1449 bp, Bacteroides fragilis
  source 1..1449: mol_type = genomic DNA
atgggacaatcaataaagaataacttcctactgaatctaagtactacaattactgggctattatttccactaataacttttccctatgcttcacgtatattaatggcagatggtatagggcaagtacaatttttccaatccataattgactatgtctctctttgtactgctttgggaatccctttgtatgccgttagagaaattgcaagaataagggataataaggaattaagaagtagaacaacaatcgaaatactattgcttcatgctattttgactttagtcggatatatagttgtttttatacttgctaagaccgttgccaaaatagaaatagatgcttctcttttctttttgttaagtacaactttattttttaacacgataggagttgcatggttttatcaggcaattgaagattttaaatacataactttacgttccttatttgtaaggatactatctttagtggccttatttatctttgttaagaccaagcaagatctgttttattatgctggaatattagttattggtactgttggtaataatatatttaatttttttcgattacgtaaatatataaaacttagtaaaggcgaatttaagcgtttgaatctgttgaggcatttaatacctgcacttaaaatttttatattaaatttggttataagtatttatgtgaatcttgattctgtgatgcttggatttcttaaaaatgaagaatcagttggttattatgctgctgctactcgacttacaaaagctattttgggtatagtttcttcattaggagctgttttattacctcgttttagtaatatgataactaatggtcaaaaagaagaattccaattattggcaaataaagctgctagttttacaattgctttaagtcttcctatgagtgtgggacttatttttatggcagcacctattatacatattttttgtggaaatggttttgaaccttctattttgacattaaaattagttgcacctattgttttatttattggattatctggaattattggaatgcaaatattgtatccgcaagggagagaaaagtacgtaattatatctactatggtaggagcatgtataaatctccttataaactatttactaataccccaatatgggcagtatggagcggcgcttgggactgttatcgcagaatttatggtaacggttataatgattttgctgggaagaaaatatttacctataaatattctctccaagcagaatttacactatctaataggttctatagtgatatctattctattagcttttttgtttgtcttccctttacatgaagtaaattatttattgataggtatactgttatctgttattgtttattacgcatatttattaatgataaaagataccctcgcattacaactaaagaaattattactttcaatatttaaacaatga

SEQ ID NO: 96 - protein, 482 aa, Bacteroides fragilis
MGQSIKNNFLLNLSTTITGLLFPLITFPYASRILMADGIGQVQFFQSIIDYVSLCTALGIPLYAVREIARIRDNKELRSRTTIEILLLHAILTLVGYIVVFILAKTVAKIEIDASLFFLLSTTLFFNTIGVAWFYQAIEDFKYITLRSLFVRILSLVALFIFVKTKQDLFYYAGILVIGTVGNNIFNFFRLRKYIKLSKGEFKRLNLLRHLIPALKIFILNLVISIYVNLDSVMLGFLKNEESVGYYAAATRLTKAILGIVSSLGAVLLPRFSNMITNGQKEEFQLLANKAASFTIALSLPMSVGLIFMAAPIIHIFCGNGFEPSILTLKLVAPIVLFIGLSGIIGMQILYPQGREKYVIISTMVGACINLLINYLLIPQYGQYGAALGTVIAEFMVTVIMILLGRKYLPINILSKQNLHYLIGSIVISILLAFLFVFPLHEVNYLLIGILLSVIVYYAYLLMIKDTLALQLKKLLLSIFKQ

SEQ ID NO: 97 - DNA, 1305 bp, Bacteroides fragilis
  source 1..1305: mol_type = genomic DNA
atgactagtacttctttctttattattaaggctatatcttttatagtatttgtttataatttagcgagttctgattggagtaatctttctcgagtattgttatttataatttcgttttttgtctttttattagtaacaaatgtatatcaaagaaaagtaaaaatagtaacactaggttatttggtctatttctttttggcttttttagctcaaaatgtaggtccatatgcaattatttacaattctcacatacagaatttgatacagaggtttcctaatatttgtgtaccaaatgatcattacactttatactttcttatttttatttttgtttcaactgttatttatatttatcttttatgtattgataaaatttctattgagcagaaagtatgggtatacatgcctacaagacaagaagctcgaaaaataatgatggcattttttataatcttgttgcctatatggatattagggaataggtcctatgctacaattctagttccttttacaagttattttatcatttctatttggaaatatccttttacaaggaaaatagtagtatttattattggttttattgcatcggtattgatccttatttcgcaattattttctcggtttatattcgttcaatatgtgttcccttttattttaatttggtttttgaataatggattgctattttcaagaaaagaaaaaataagttataaagttattggcttttttctattgttttgttctttagcgattttgtatggtatcatttcagagatgatgaagttaaatgcaaattttgatggaaactatactttcaatgatatgtttgaagtattaagtgagccgcaattattgcaaaattggttatctaggcaaacatatcgtatttttgatatttggtcacatttgggggggaatataatagattatattgaccagaaaggatatttatggggaattacatatgtaaagagctttgcacctatattaggttttgattatgtaagccttccattaatttcagcaaatatgattggtgcagattatgctcaacctggtttggtggcagaagggtacgctaattggggaatatatggggctataattaatatgatatttgtttttttcgttgcagagttattgtttgattattttttacgaaaacaatctaccttaaatttgttactttatattggaactttttcccaggtattactagatggggggaccattaattctattattttcatgtctatattttgtttcttaacttgtttgtttaatgccaaaatcaaattttaa

SEQ ID NO: 98 - protein, 434 aa, Bacteroides fragilis
MTSTSFFIIKAISFIVFVYNLASSDWSNLSRVLLFIISFFVFLLVTNVYQRKVKIVTLGYLVYFFLAFLAQNVGPYAIIYNSHIQNLIQRFPNICVPNDHYTLYFLIFIFVSTVIYIYLLCIDKISIEQKVWVYMPTRQEARKIMMAFFIILLPIWILGNRSYATILVPFTSYFIISIWKYPFTRKIVVFIIGFIASVLILISQLFSRFIFVQYVFPFILIWFLNNGLLFSRKEKISYKVIGFFLLFCSLAILYGIISEMMKLNANFDGNYTFNDMFEVLSEPQLLQNWLSRQTYRIFDIWSHLGGNIIDYIDQKGYLWGITYVKSFAPILGFDYVSLPLISANMIGADYAQPGLVAEGYANWGIYGAIINMIFVFFVAELLFDYFLRKQSTLNLLLYIGTFSQVLLDGGTINSIIFMSIFCFLTCLFNAKIKF

SEQ ID NO: 99 - DNA, 45 bp, synthetic construct
  source 1..45: mol_type = other DNA
tcataaaaaatttatttgctttgtgagcggataacaattataata

SEQ ID NO: 100 - DNA, 14 bp, synthetic construct
  source 1..14: mol_type = other DNA
aggaggatatacat

SEQ ID NO: 101 - DNA, 12764 bp, synthetic construct
  source 1..12764: mol_type = other DNA
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggaatgattttttataaaagcaaaagattagcattttttttaacttcagatattgttttaattttacttagcgtttatctggctttttctttgagatttagtggagatattccgagtattttttatcatggtatgatggtttctgctattattttgcttgttttaaaactttcatttttgtttgtttttagaatttataaagtagcttggagatttttttctctcaatgaagcaagaaagatttttatcgctttgcttttagctgagttttgtttttttcttattttttatttttttagtgatttttttaatccttttccaagaagtgctattgtgatagattttgttctttcttatatgtttataggtactttaagaattagcaaaagaatgcttgtggattttaaaccttctagaatgaaagaagaagaaactccttgtattgtagtaggggcaacttctaaggctttgcatttgttaaaaggtgcaaaagaaggttctttagggctttttcctgtaggcgtagttgatgcgagaaaagagcttatagggacttattgtgataaatttattgtagaagaaaaagaaaaaataaaatcttatgtagaacaaggggtaaaaactgccattattgctttaagacttgaacaagaagagcttaaaaaactttttgaagaacttgtagcttatggtatttgcgatgtaaaaatattttcttttacaagaaacgaagcaagagatatcagtatagaagacttgcttgctagaaaaccaaaagatttagatgatagtgctgtggcggcttttttaaaagataaggtagttttggtaagtggagcaggtggaactataggcagtgaactttgtaagcaatgtattaaatttggtgctaagcatcttatcatggttgatcatagtgagtataatctttataagatcaatgatgatttaaatttatataaagaaaaaattactcctattttactgagtattttagataagcaaagtttagatgaggtattaaaaacttataaacccgagcttattttacatgcagccgcttataaacatgtgcctctttgcgaacaaaatccacattcagcagtaatcaataatattttaggaactaaaattttatgcgacagtgctaaagaaaacaaagtagctaaatttgtgatgataagtacagataaagcagtacgaccaacaaatattatgggttgcactaagagagtttgcgagctttatactttaagtatgagtgatgaaaattttgaagttgcttgtgtgcgttttggtaatgttttaggttctagtggtagtgtgataccgaaatttaaagcacaaattgccaataatgagcctttaactttaacgcaccctgatatagtgcgttattttatgcttgtggctgaggcagtgcaacttgttttacaagctggagctatcgcaaaagggggagaactttttgttttggatatgggtaagcctgtgaaaatcatagatttagctaaaaaaatgcttttactttctaatcgcaatgatttagaaattaaaatcacaggcttaagaaaaggtgagaagctttatgaagagcttttgattgatgaaaatgatgctaaaacacaatatgagagtatttttgtagcaaagaatgagaaggttgaccttgattggcttaataaagagatagaaaatttacaaatatgtgaagatatttcagaggctttattaaagattgtacctgaatttaaacacaataaagaaggtgtataaaggaggatatacatatgaaaatacctttttcaccaccttatattgacgaggcggtcatcaacgaagtcgttgattcgttacgttccggttggatcacatccggtccgaaagtgaaggctttggaggaagaaattaaatctttctcaggagcaaaagaagtgctttgtgtcaattcctggacatcaggagctattatgatgttgcgttggctgggagtgaaggagggcgatgaagtgattgttcctgcctatacttacagtgcaacggcattggctgtgctccatgccggagccaaaccggtaatggtggattccggaactgattttaatatttcggtggaggctgttcgtaaagctattactcctaaaacaaaggcgattatacccgtagatattgctggttttccctgcgattatgaaagaatcatggcactggtgcaggaaccagaaatggtaaaactgttccgttcggaatctcctgtgcaagaaaaattgggacgcatcttggtgatgaatgatgctgcccactctttgggagcacgttacagtagccgtcagcgtaccggttgtgaaactgatgtggctatcttttcacttcatgccgtgaaaaatgtgactactgccgaaggtggggccatttgcctgaatctgcctaagccgttcgataatacggagttgtataaagagctacggatgacaagcttgaactgccagaccaaggatgctttttcgaaatctaaagccggaggatggcgttatgatattgtcggtttcgggatgaaaatcaatatggccgatgtaaatgccgccataggactggcacagatacgagaatatccggaattgctgaaagaacgaaagcgggtgtttaatgcttatagcgatgctttttcggcttgtgactgggccatcgttccgccttctgtggatggggaaaaggaaagctcttatcacatttatgctttgcgtattaaggactttaccgaagaacaacgcgaccggatgattgatgaaatcgctaaaagtgaagtggctgtcaatgtgcactttataccgatgccgatgttgtcgttcttcaaatcgatgggatatgatataaaggattatccacaggcttatcagaactttaagagcgagatatcattgccgatatatccccaattggatagtgaaaaattgaatttcattattgaaaccgtaaaggcggcctatgcgaccgtaattgcagaaaaccgacaccaccaccaccaccactagaggaggatatacatttgatccgtttttttgatatcgttttttcccttttgggaattcttctgctttctcccgtttttcttcttctttatatagccatttgtctcgaaagtaaaggaggtggtttctataaacagctccgtgtgggaaggtacggaggggatttttatgtttataaatttcgttcgatgcgtgtaggggctgataaaaaggggttgataacggtaggagggagagatccgagaataactcgtaccggatacctgatccggaagtataaattggatgaattgcctcagttgtttaacgtactgaaaggagatatgagtctggtagggccccgtcctgaggttcgtaagtatgtggatttatataccgatgaacaaaagaaagtattgagtgtacgtccggggattacggattatgcttctattgagtatgtagatgaaaatatgattcttggagaggcttcggatcctgacagagcatatatagaacagattatgcccgataagatacggtataatatgaaatatatctgtaatcgttcggtgaaggaatattttaaaattatatttctgacattttggagcatcattcgttaggagctcaggaggatatacatatgaaattggctgtcatttcttctatctacaaaaatgattcgctttctaaaataaagcaatccttacaaagtttgttgaatcaagaaaatgttacttgtgatatttatttatattgtgatggtcctgtttcgattgatattgtgcatttcttagactcatttgatcaaaactcactttatatatttaagcaagaacaaaatagaggattggctcatgcactaaatgaattattgaatatcgttcttacaaaagaatacgaatatattgctcgtatggatgctgatgatatttccatgccggaacgttttgtgaagcagatcgcatttatgaattcacatccggatattgattgccttggtacttgggcaatagaaattgatgatgacggaaaagaatattttcgaaaaaaaatgccgataactcatgaagaatgtttggaactgtttaaaaagcgggattgtatgattcatcctactgtgatgtttcgacgttcatattttgagaaagctggtttatatcccgaagacacctactttggagaagatactatgatgtgggctaaaggttttaaaagtggatgtaaatttgccaatgtacccgaatatctgtttaagtttagattggattcaaacttctttgaaagaaggcgggggtggaaacatgcaaaatctatttatactttacgtcatcgtgttaatagaatgttagggttcggatggaaggaagattgttatgcattgctatatgctatggccaaattaatgcctaaatctattcttgacataatctataaaacagtacgttgaaggaggatatacatatgaggaagatattattaacatatggagatattaaaaccataaatattggtgattatattcagagtatcgccgctaaacaattttttgatgatgataattatatatttttcaatcgtgatgaattaaagttgtataaaggggaacctgcaaaagtcataatgaatgcttggatgacttataaaccatataattggcctccttcatctcaggtttatccattatttgttgcattacatatcaattcttctgcagaaagtcgctttttgtcacatgatagtattaagtatttaaaaaagtatgaacccatagggtgccgcgattatcatacaatgaatattttaaaaggaaaaggtgttaatgcttatttttcgggatgtcttactactactttgggtaaaacttataaatataatggaaaacgtgaggggatatacattgttgatccactttcatacatgcctaatgggaataatttttttgaaataatgaaagctgttgtacaaactgttttttatatgaagcctgtgctaaagattcttagaaattataaaaagaataatagatttactattaatatatccaaagtaggaataggacgtttattgttaataaccaagtcttatttgcttttgagaaagctagtagttcctgacgtattatacaatgcaatatttataacacaatttaatatgtctaatgaatattcttctgaatcagaaagatttgctcgggctgatgaattattaactaaaatggcctcagctcaatatgtgataacttctagaatccactgtgctcttccttgcttaggctttgagacccctgtggtttatattagaaatctatcagaaagcaagaagagcacttgtcgattagggggacttgaatctctatttaatgtgataactgtgaagggggaaaatgttacttccaatttttttgatggactatttaaacgagattcttcttttaagaataaaattgattttgtagaatatagagatcgattaattaatatatgtaataaatttatgatgtcatgaaggaggatatacatatgagggatggaaagcccattgaaatactgcatattgtagggactagacctgttggaggaattggtgctttattgaaaaatataaacacttctattgatttaaataaattccattttacatatgtgttttctgccgatagtaatattggagattttgataattatgtgagaaaattaggtagcgatattgttgtattcccttcatatcatttgaaatatttatttttatatttaaaggtgattttctgtttttataagagaaatgcaaaaaaatatgatataattcatgtacatagtgctaatacaggagtattagaccttttatttgcaaagatatatggaatacggataaggattttacatagtcacagtacaaaatattctagtaaaaaaataaggtccattagaaattattttttgcaatttcctacaatatatttagcaaacacttattttgcatgtggatacaaagctgctgaattcctttttggaaaaaagagacttgaaaaagtttatattattcataatgcaattttttctaaaaaatttatttataatgcaactgttagagagagagttagagttgagttgaaaatagagaatgactgtttactgttggggcatatcggtaactttacgaaagaaaagaatcatggttttttaatagatatattggaagaattattgaatattaatgacaatgttaagttgctgttagtaggggatggccaacttagaagtgaaatagaggaaaaagtaaaattaaaaagattgcagaagtatgtgtgctttttaggtcggagaactgatatatcagaattattacaagctatggattttttaatttttccttctttttttgaaggtctaccagtgactctgattgaagcgcaagcttctggtttaagatgtgtcgtttcggatcgtattactttggaaacaaagataacgaataatataagttatcttaatctcaatcgagatccttcaacttgggctgaagttgtttggaagttgtttgaagataaaacttttatcaggaaagatacgtctaaagaaatatgtgaatcaggatatgatatacatttagagagtgaatatctcgaaagaatatatttaaatcttttacataaatgaaggaggatatacatatgaccaaatacgaaaaaatccaacaagatttagttgaatcaccaaaaacatggttgataaccggtgttgctggttttattggctccaatttactagaaaaactactaaagcttgatcaaaccgtggtggggttagataactttgccacgggtcatcaacataacttagatgaagttaaaagtttagtaacagaagcccaatggcaacgatttacctttattgaaggcgatatacgtaatcctgagacctgtgtagcagcggttcaaggtgtagactatgtattacaccaagcggcactagggtctgtgccccgttcaattaatgatcctattactacaaattccgctaacatcactggttttttaaacatgttagtagcagccaaagatcagcaagtgaaaagctttacttatgccgcctcaagttcgacttatggtgatcaccctgcattgccgaaggtagaagaaaatattggcaacccgttatccccctacgccgtaaccaaatacgtaaatgaactttatgcgaatgtatttgcgcggacttatggttttaaaagtattggactgcgttattttaatgtgtttggaaaacgccaagatccaaatggtgcctatgcggctgttattccaaaatggacagcagcaatgattcaaggtgatgttatttatattaatggtgacggcgagactagtcgcgatttctgttatatagctaatacagtgcaaatgaatattcttgccgccaccgcaccagaagaagcaaaagacaagatttacaatgttgcagtaggagacagaacaaccttaaacgatctattctctgccattaaattggcattatcgaataatactgttgttgtttctcaagaacctatttatagagagtttcgtactggtgatgtaaggcattctcaggctgatgttaataaagctaagcagtatttaggctatgcgccgtcgcaccgtattctagatggaatcaacgaagcaatgccatggtatataagctttataaatgataaaaaataaaggaggatatacatatgaaaaaaaaatatgactatctaattgtcggagccggactttatggttctgtttttgcatataaagcgcaacggaatgggaaaaaatgtttgcttattgacaaacgtccacacaatggaggaaatatttattgcgaaaatattgagggtataaatgtacacaaatatggtgcacatatttttcatacttctaataaagaggtttgggactttgtaaatgccatagtggaatttaaccgctatactaattcccctattgccaattataaaggcaaattatataatttaccttttaatatgaatactttttatgctttatgggggacaaaaactccagaggaagcaaaaatgaagatagaagagcaaaggagagaagcggggattcttacgccgaagaatctagaagaacaggctatctctttagtaggaaaggatatttatgaaatattgataaaaggatatactgagaaacagtgggggcggaaagcaactgaacttcctgcttttataatcaagcgtttacctgtcagatttacttttgataataattactttaatgataaatatcaggggattcctgttggtggctataataaattgataaatggtttattagatggtattgaagttagaacaaatacagacttttttcaaaataaagaatattttaaaggtcttgcggataaaattctatttactggcaaaattgatgaatattataattatcaatttgggagattggaatatcgtactgttgaatttgaaacacaagtgcttgattgtgagaattatcaagggaatgcagttgtgaattatactgaaagggaagtgccttatactcgtattatagagcataaacattttgaatttggaacacagcccaaaaccgtaatttccaaagaatattcttcggaatggaaagatggtagtgaacccttttatcctgttaatgatgagaggaataattctttgtatttggagtataagaaattggtcgatagagagaagaatgttttgtttggagggagattagctgagtataagtattatgatatgaatgtgattgtggaaaaagttataaatagtgacttatgaaggaggatatacatatgaagatatttgctgtagttgtgacctataatcgtttagcattactaaaaaaggtcatagatttacttaaaagacagacacgtcaattagatagtatagttatagtaaataatggctctactgatggaacaaaagaatggttagaacaggagaaaaatataacattaataaatcaatctaattctggtggggctggtggattcgaaacgggggtaaaatatgcatatgagaatgaggcagattggatttggatgatggatgatgatgtttttcctaatatagattgtttggaaaaactcttagggtggacgagtatttctcaatgtatccaaccacgtaggtattattcggatgatgtagaagtaaattttgaacaatggttggatccaattacttatagtaaatttggttattggcaagaaaaatcatttaataatggtaaaaagttttgtgctattaatgtaggctgttttgaagggatgtttgtttcgaaaagcattgttaataaaataggatttcctgataagcgtttttttatagcggaagatgatacaatatatgggtttgcagctagtttttataccaacgtaattttagtatctgatgctatcatggttcgtgctagaagatcatcggatcgtagcgttagcccaatgtatacttattatgcatgtcggaattttcatttagtatatgagtcacttaatttattattggataagaaaaataggtttttatatattaaatatatttatcaatttgttcatcagatttatttatctatttttttatatgataataagatgaaacacttaatatctgtttttcgtggcttttatgattgctttcgtaaaaaaaatggagctacctattaaaggaggatatacatatgggacaatcaataaagaataacttcctactgaatctaagtactacaattactgggctattatttccactaataacttttccctatgcttcacgtatattaatggcagatggtatagggcaagtacaatttttccaatccataattgactatgtctctctttgtactgctttgggaatccctttgtatgccgttagagaaattgcaagaataagggataataaggaattaagaagtagaacaacaatcgaaatactattgcttcatgctattttgactttagtcggatatatagttgtttttatacttgctaagaccgttgccaaaatagaaatagatgcttctcttttctttttgttaagtacaactttattttttaacacgataggagttgcatggttttatcaggcaattgaagattttaaatacataactttacgttccttatttgtaaggatactatctttagtggccttatttatctttgttaagaccaagcaagatctgttttattatgctggaatattagttattggtactgttggtaataatatatttaatttttttcgattacgtaaatatataaaacttagtaaaggcgaatttaagcgtttgaatctgttgaggcatttaatacctgcacttaaaatttttatattaaatttggttataagtatttatgtgaatcttgattctgtgatgcttggatttcttaaaaatgaagaatcagttggttattatgctgctgctactcgacttacaaaagctattttgggtatagtttcttcattaggagctgttttattacctcgttttagtaatatgataactaatggtcaaaaagaagaattccaattattggcaaataaagctgctagttttacaattgctttaagtcttcctatgagtgtgggacttatttttatggcagcacctattatacatattttttgtggaaatggttttgaaccttctattttgacattaaaattagttgcacctattgttttatttattggattatctggaattattggaatgcaaatattgtatccgcaagggagagaaaagtacgtaattatatctactatggtaggagcatgtataaatctccttataaactatttactaataccccaatatgggcagtatggagcggcgcttgggactgttatcgcagaatttatggtaacggttataatgattttgctgggaagaaaatatttacctataaatattctctccaagcagaatttacactatctaataggttctatagtgatatctattctattagcttttttgtttgtcttccctttacatgaagtaaattatttattgataggtatactgttatctgttattgtttattacgcatatttattaatgataaaagataccctcgcattacaactaaagaaattattactttcaatatttaaacaatgaaaggaggatatacatatgactagtacttctttctttattattaaggctatatcttttatagtatttgtttataatttagcgagttctgattggagtaatctttctcgagtattgttatttataatttcgttttttgtctttttattagtaacaaatgtatatcaaagaaaagtaaaaatagtaacactaggttatttggtctatttctttttggcttttttagctcaaaatgtaggtccatatgcaattatttacaattctcacatacagaatttgatacagaggtttcctaatatttgtgtaccaaatgatcattacactttatactttcttatttttatttttgtttcaactgttatttatatttatcttttatgtattgataaaatttctattgagcagaaagtatgggtatacatgcctacaagacaagaagctcgaaaaataatgatggcattttttataatcttgttgcctatatggatattagggaataggtcctatgctacaattctagttccttttacaagttattttatcatttctatttggaaatatccttttacaaggaaaatagtagtatttattattggttttattgcatcggtattgatccttatttcgcaattattttctcggtttatattcgttcaatatgtgttcccttttattttaatttggtttttgaataatggattgctattttcaagaaaagaaaaaataagttataaagttattggcttttttctattgttttgttctttagcgattttgtatggtatcatttcagagatgatgaagttaaatgcaaattttgatggaaactatactttcaatgatatgtttgaagtattaagtgagccgcaattattgcaaaattggttatctaggcaaacatatcgtatttttgatatttggtcacatttgggggggaatataatagattatattgaccagaaaggatatttatggggaattacatatgtaaagagctttgcacctatattaggttttgattatgtaagccttccattaatttcagcaaatatgattggtgcagattatgctcaacctggtttggtggcagaagggtacgctaattggggaatatatggggctataattaatatgatatttgtttttttcgttgcagagttattgtttgattattttttacgaaaacaatctaccttaaatttgttactttatattggaactttttcccaggtattactagatggggggaccattaattctattattttcatgtctatattttgtttcttaacttgtttgtttaatgccaaaatcaaattttaaagctctaattagctgagcttggactcctgttgatagatccagtaatgacctcagaactccatctggatttgttcagaacgctcggttgccgccgggcgttttttattggtgagaatccaagc

(19) United States
(12) Patent Application Publication
(10) Pub. No.: US 2025/0270499 A1
(43) Pub. Date: Aug. 28, 2025

(54) RECOMBINANT PRODUCTION OF BACTEROIDES FRAGILIS CAPSULAR POLYSACCHARIDE A IN ESCHERICHIA COLI

(71) Applicant: The University of North Carolina at Charlotte, Charlotte, NC (US)

(72) Inventors: Jerry M. Troutman, Charlotte, NC (US); Beth A. Scarbrough, Charlotte, NC (US)

(21) Appl. No.: 19/062,002

(22) Filed: Feb. 24, 2025

Related U.S. Application Data
(60) Provisional application No. 63/557,286, filed on Feb. 23, 2024.

(51) Int. Cl. C12N 1/20; C08B 37/08; C12N 9/00; C12N 9/04; C12N 9/10; C12N 9/90; C12R 1/19

(52) U.S. Cl. CPC C12N 1/20; C08B 37/0072; C12N 9/0006; C12N 9/1051; C12N 9/1096; C12N 9/90; C12N 9/93; C12R 2001/19; C12Y 101/01095; C12Y 204/01001; C12Y 206/01021; C12Y 501/03007; C12Y 504/99004; C12Y 604/01001

(57) ABSTRACT

Provided are methods for producing polysaccharides in bacteria by expressing in a bacterium one or more coding sequences selected from the group consisting of a pglF dehydrogenase coding sequence, a wbpP UDP-N-acetyl-d-glucosamine C4 epimerase coding sequence, a wcfR aminotransferase coding sequence, and a wcfS phospho-glycosyltransferase coding sequence, a wcfQ glycosyltransferase coding sequence, a wcfO pyruvyltransferase coding sequence, a wcfP glycosyltransferase coding sequence, a wcfM UDP-galactopyranose mutase coding sequence, a wcfN glycosyltransferase coding sequence, a wza polysaccharide export protein coding sequence, a wzx fippase coding sequence, a wzy polymerase coding sequence, and a wzz coding sequence, wherein at least one of the coding sequences is heterologous to the bacterium. Also provided are expression cassettes with one or more of the disclosed coding sequences, recombinant bacteria that harbor one or more of the expression cassettes, and methods for producing immunogenic compositions using the polysaccharides produced by the recombinant bacteria.

### CROSS REFERENCE TO RELATED APPLICATIONS

This application claims the benefit of U.S. Provisional Patent Application Ser. No. 63/557,286, filed Feb. 23, 2024, the disclosure of which is incorporated herein by reference in its entirety.

### GOVERNMENT INTEREST

This invention was made with government support under R01 GM123251 awarded by the National Institutes of Health (NIH). The government has certain rights in the invention.

### REFERENCE TO SEQUENCE LISTING XML

The Sequence Listing XML associated with the instant disclosure has been electronically submitted to the United States Patent and Trademark Office via the Patent Center as a 125,559 byte UTF-8-encoded XML file created on Feb. 24, 2025 and entitled “1276_13_2_ST26.xml”. The Sequence Listing submitted via Patent Center is hereby incorporated by reference in its entirety.

### TECHNICAL FIELD

The presently disclosed subject matter relates to compositions and methods for producing polysaccharides in bacteria. In some embodiments, the compositions and methods are based on expression of a plurality of coding sequences in the bacteria, which in some embodiments are coding sequences at least one of which is heterologous to the bacteria.

### BACKGROUND

Polysaccharides have drawn considerable attention as potent and renewable sources of biological polymers. These polymers are ubiquitous among prokaryotes, and many possess unique chemical structures that bestow distinct physical and chemical attributes. Diverse physicochemical properties present advantages of applying these polymers in various settings such as wound healing, pharmaceuticals, cosmetics, and food industries.

A major hindrance to the application of many bacterial polysaccharides is the inability to isolate or synthesize them in large quantities. This is due to complex growth requirements of bacterial hosts, complicated and laborious isolation procedures, and difficulty of genetic manipulation of hosts to aid in isolation procedures and improve yield. Thus, recombinant polysaccharide production in model strains of E. coli have been underway, including multiple Streptococcus pneumoniae capsule serotypes, and the N-linked glycan from Campylobacter jejuni (Feldman et al., 2005; Kay et al., 2016; Nothaft et al., 2016; Kay et al., 2022). In both instances, these recombinant polysaccharides were employed for the development of glycoconjugate vaccines.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter relates to methods for producing polysaccharides in bacteria. In some embodiments, the methods comprise expressing in the bacterium a plurality of coding sequences, the plurality of coding sequences comprising nucleotide sequences that encode a pglF dehydrogenase gene product, a wbpP UDP-N-acetyl-d-glucosamine C4 epimerase gene product, a wcfR aminotransferase gene product, and a wcfS phospho-glycosyltransferase gene product, and optionally further comprise nucleotide sequences encoding one or more of a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, and further wherein at least one of the coding sequences is heterologous to the bacterium. In some embodiments, the polysaccharide is a capsular polysaccharide A (CPSA) and the bacterium expresses each of the PglF gene product, the wbpP gene product, the WcfR gene product, the WcfS gene product, the WcfQ gene product, the WcfO gene product, the WcfP gene product, the WcfM gene product, the WcfN gene product, the wzz gene product, the wzx fippase gene product, and the wzy polymerase gene product, at least one of which is heterologous to the bacterium. In some embodiments, the bacterium is an Escherichia coli (E. coli) bacterium. In some embodiments, the E. coli bacterium has reduced or absent waal biological activity. In some embodiments, the pglF gene product is a Campylobacter jejuni gene product, the wbpP gene product is a Vibrio vulnificus gene product, the wcfR gene product is a Bacteroides fragilis gene product, the wcfS gene product is a Bacteroides fragilis gene product, the wcfQ gene product is a Bacteroides fragilis gene product, the wcfO gene product is a Bacteroides fragilis gene product, the wcfP gene product is a Bacteroides fragilis gene product, the wcfM gene product is a Bacteroides fragilis gene product, the wcfN gene product is a Bacteroides fragilis gene product, the wza gene product is an Escherichia coli gene product, the wzx fippase gene product is a Bacteroides fragilis gene product, the wzy polymerase gene product is a Bacteroides fragilis gene product, the wzz gene product is a Bacteroides fragilis gene product.

In some embodiments, the polysaccharide is present in a lipid bilayer of the bacterium, optionally wherein the lipid bilayer is part of the bacterium's cell wall.

In some embodiments, the polysaccharide is a CPSA, optionally an aggregate of two or more CPSA tetrasaccharide monomers.

In some embodiments, the plurality of coding sequences are transcribed to produce a polycistronic messenger RNA (mRNA) molecule in the bacterium. In some embodiments, the polycistronic mRNA molecule comprises a ribosome-binding site (RBS) operatively linked to a translation initiation codon of each of at least two of the plurality of coding sequences, optionally wherein the polycistronic mRNA molecule comprises a plurality of ribosome-binding sites (RBSs), with an RBS present 5′ to each of at least two, optionally 5′ to each, translation initiation codon present in the polycistronic mRNA, wherein at least one RBS is located 3′ to a translation termination codon of a first coding sequence and 5′ to a translation initiation codon of a second coding sequence present on the polycistronic mRNA molecule.

In some embodiments, the bacterium does not express a functional waal O-antigen ligase gene product.

The presently disclosed subject matter also relates in some embodiments to bacteria comprising a plurality of coding sequences, the plurality of coding sequences comprising nucleotide sequences that encode two or more of a pglF dehydrogenase gene product, a wbpP UDP-GlcNAc C4 epimerase gene product, a wcfR aminotransferase gene product, a wcfS phospho-glycosyltransferase gene product, a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, wherein at least one of the plurality of coding sequences is heterologous to the bacterium. In some embodiments, the bacterium is an E. coli bacterium. In some embodiments, the pglF gene product is a Campylobacter jejuni gene product; and/or the wbpP gene product is a Vibrio vulnificus gene product; and/or the wcfR gene product is a Bacteroides fragilis gene product; and/or the wcfS gene product is a Bacteroides fragilis gene product; and/or the wcfQ gene product is a Bacteroides fragilis gene product; and/or the wcfO gene product is a Bacteroides fragilis gene product; and/or the wcfP gene product is a Bacteroides fragilis gene product; and/or the wcfM gene product is a Bacteroides fragilis gene product; and/or the wcfN gene product is a Bacteroides fragilis gene product; and/or the wza gene product is an Escherichia coli gene product; and/or the wzx flippase gene product is a Bacteroides fragilis gene product; and/or the wzy polymerase gene product is a Bacteroides fragilis gene product; and/or the wzz gene product is a Bacteroides fragilis gene product. In some embodiments, the plurality of coding sequences are transcribed in the bacterium to produce a polycistronic messenger RNA (mRNA) molecule in the bacterium. In some embodiments, the polycistronic mRNA molecule comprises a ribosome-binding site (RBS) operatively linked to a translation initiation codon of each of at least two of the plurality of coding sequences, optionally wherein the polycistronic mRNA molecule comprises a plurality of ribosome-binding sites (RBSs), with an RBS present 5′ to each of at least two, optionally 5′ to each, translation initiation codon present in the polycistronic mRNA, wherein at least one RBS is located 3′ to a translation termination codon of a first coding sequence and 5′ to a translation initiation codon of a second coding sequence present on the polycistronic mRNA molecule. In some embodiments, translation of the polycistronic mRNA molecule in the bacterium results in production of a polysaccharide in the bacterium, optionally wherein the polysaccharide is present in a lipid bilayer of the bacterium, further optionally wherein the lipid bilayer is part of the bacterium's cell wall. In some embodiments, the polysaccharide is a CPSA, optionally an aggregate of two or more CPSA tetrasaccharide monomers.

In some embodiments, the presently disclosed subject matter relates to methods for producing immunogenic compositions. In some embodiments, the methods comprise mixing an antigenic molecule with a capsular polysaccharide A (CPSA), wherein the CPSA is produced by a bacterium as disclosed herein.

In some embodiments, the presently disclosed subject matter also relates to expression cassettes. In some embodiments, the expression cassettes comprise a plurality of coding sequences derived from at least two different bacterial species, wherein the plurality of coding sequences are selected from the group consisting of a pglF dehydrogenase gene coding sequence, a wbpP UDP-N-acetyl-d-glucosamine C4 epimerase gene coding sequence, a wcfR aminotransferase gene coding sequence, a wcfS phospho-glycosyltransferase gene coding sequence, a wcfQ glycosyltransferase gene coding sequence, a wcfO pyruvyltransferase gene coding sequence, a wcfP glycosyltransferase gene coding sequence, a wcfM UDP-galactopyranose mutase gene coding sequence, a wcfN glycosyltransferase gene coding sequence, a wza polysaccharide export protein gene coding sequence, a wzx fippase gene coding sequence, a wzy polymerase gene coding sequence, and a wzz gene coding sequence.

In some embodiments, the expression cassette comprises a combination of coding sequences selected from the group consisting of:
pglF, wcfR, and wcfS;
pglF, wcfR, wcfS, and wcfQ;
pglF, wcfR, wcfS, wcfQ, and wcfO;
pglF, wcfR, wcfS, wcfQ, wcfO, wbpP, and wcfP;
pglF, wcfR, wcfS, wcfQ, wcfO, wbpP, wcfP, wcfM, and wcfN; and
pglF, wcfR, wcfS, wcfQ, wcfO, wbpP, wcfP, wcfM, wcfN, wzx, and wzy.

In some embodiments, the expression cassette is operatively linked to a promoter such that transcription from the promoter results in the production of a polycistronic mRNA that is translatable to produce polypeptides encoded by the coding sequences. In some embodiments, the polycistronic mRNA molecule comprises a ribosome-binding site (RBS) operatively linked to a translation initiation codon of one or more of, optionally each of, the plurality of coding sequences, wherein at least one RBS is located 3′ to a translation termination codon of a first coding sequence and 5′ to a translation initiation codon of a second coding sequence, wherein the first coding sequence and the second coding sequence are adjacent to each other on the polycistronic mRNA molecule, the first coding sequence being present 5′ to the second coding sequence. In some embodiments, the polycistronic mRNA molecule is operably linked to a promoter that is active in the bacterium, optionally wherein the promoter is an inducible promoter, optionally an isopropyl-β-d-thiogalactopyranoside (IPTG)-inducible promoter, further optionally wherein the promoter is a T5 promoter.

Accordingly, it is an object of the presently disclosed subject matter to provide recombinant nucleic acids that include coding sequences from different organisms that can be employed for producing polysaccharides including but not limited to CPSA in bacteria.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following Description, Figures, and EXAMPLES.

### BRIEF DESCRIPTION OF THE FIGURES

FIG. 1 is a representation of the CPSA biosynthesis pathway and structure in Bacteroides fragilis, and a reconstitution of its biosynthesis in E. coli. The previously determined structure of the CPSA unit oligosaccharide is highlighted in yellow.

FIGS. 2A-2C illustrate Recombinant UDP-AATGal and UDP-GalNAc biosynthesis in E. coli. (FIG. 2A) UDP-AATGal is produced from UDP-GlcNAc by PglF from Campylobacter jejuni and WcfR from B. fragilis. (FIG. 2B) UDP-GalNAc is produced from UDP-GlcNAc by WbpP from Vibrio vulnificus. (FIG. 2C) Schematic of CPSA biosynthesis in E. coli utilizing PglF and WbpP.

FIG. 3 depicts the LCMS identification of individual BPP-linked CPSA intermediates recombinantly produced in E. coli. Each BPP-linked CPSA intermediate was identified in lysates of E. coli DH5a cells recombinantly expressing genes from the CPSA biosynthesis operon using ESI-LCMS and SIM. Descriptions of plasmids used to generate individual intermediates in E. coli and the exact mass of each intermediate can be found in Table 4. Only chromatograms generated from SIM of the anticipated intermediates were overlayed in FIG. 3 (see also FIG. 11). 1: BP; 2: BPP-mono; 3: BPP-Di; 4: BPP-PyrDi; 5: BPP-Tri; 6: BPP-tetra.

FIG. 4 shows Both flippase (Wzy) and polymerase (WzX) from B. fragilis may be required for polymerization of CPSA oligomers in E. coli. SDS-PAGE (left panel) and anti-CPSA western blot (right panel) of E. coli DH5a cell lysates expressing an empty vector control (pQE-80L), pBAS16 (−wzx, −wzy_{Bf}), pBAS17 (+wzx, +wzy_{Bf}), pBAS18 (+wzx_{Bf}), or pBAS19 (+wzy_{Bf}). The left panel is a Ponceau stained SDS-PAGE gel of normalized cell lysates. The right panel is an anti-CPSA western blot of cell lysates. Genes assembled in plasmids are listed in Tables 4 and 5. CPSA positive (+) indicates that all genes are present to produce CPSA oligosaccharide.

FIG. 5 shows confocal microscopy of cells expressing CPSA polymers (pBAS17; right panel) compared to an empty vector control (pQE-80L; left panel).

FIGS. 6A-6C show that CPSA polymers were detected in both aqueous and organic fractions of E. coli pBAS17 lysates. Wild-type (FIG. 6A) and a ΔwaaL mutant (FIG. 6B) of E. coli MG1655 pBAS17 were evaluated for the presence of CPSA polymers in aqueous, organic, and insoluble cell fractions. Cell fractions were separated using SDS-PAGE and anti-CPSA antiserum. Lanes designated with an “A” are aqueous fractions, lanes designated with “O” are organic fractions, and lanes designated with “I” are insoluble cell fractions. (FIG. 6C) SDS-PAGE and anti-CPSA western blot of whole cell lysates.

FIG. 7 shows the whole cell western blot of E. coli MG1655 expressing pBAS16 or an empty vector control. The left panel shows a whole-cell dot blot of wild-type (WT) and mutant MG1655 expressing pBAS16 (−wzx, −wzy). The right panel shows PRO-Q™ Emerald staining of LPS in normalized, pronase-treated lysates. The lower (blue) arrow indicates lipid A core. The upper (white) arrow likely indicates CPSA oligomer(s) ligated to LOS.

FIG. 8 shows CPSA surface expression was not abolished in a ΔwaaL or Δwza mutant expressing pBAS17. The left panel is a whole cell dot blot of E. coli MG1655 expressing pBAS17 (+wzx, +wzy) or an empty vector control (pQE-80L; EV). The right panel shows PRO-Q™ Emerald 300 stain of LPS from pronase-treated cell lysates. The lower (blue) arrow indicates lipid A core. The upper (white) arrow likely indicates CPSA oligomer(s) ligated to lipid A core.

FIG. 9 is a series of spectra of recombinantly produced BPP-linked CPSA intermediates identified within E. coli cell lysates. Plasmids, CPSA intermediate, and [M-H]-ion of each intermediate described in the Table above the spectra and correspond to each numbered spectrum.

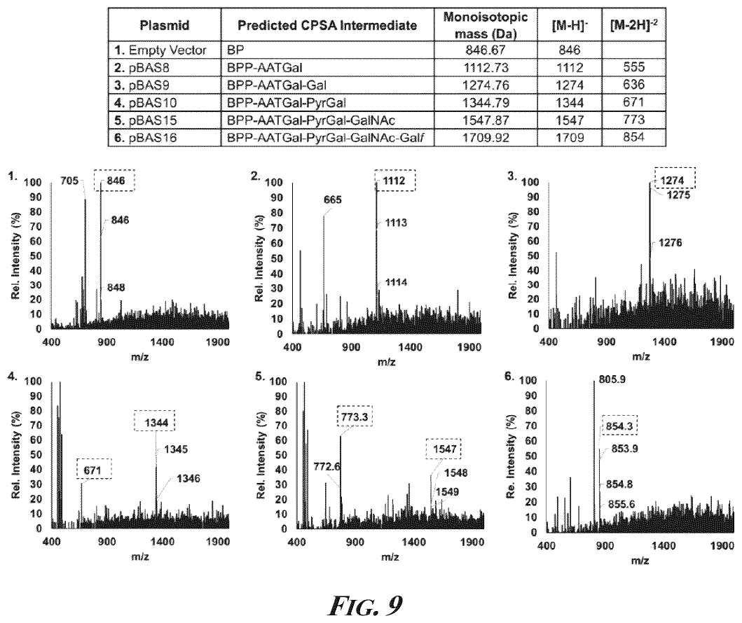

FIG. 10 shows that CPSA intermediates were not detected in E. coli expressing an empty vector control. Lysates of E. coli expressing an empty vector control were evaluated with Liquid Chromatography-Electrospray Ionization-Mass Spectrometry (ESI-LC-MS) and selected ion monitoring (SIM) for all BPP-linked CPSA intermediates. A contaminant with a m/z identical to the first CPSA intermediate (BPP-mono) is observed at 2.84 min which is inconsistent with the retention time for a BPP-linked monosaccharide. The panel at the upper right is a zoomed in region of the chromatogram at the lower left, showing less abundant contaminants.

FIG. 11 is a series of graphs the LC-MS with SIM of recombinant E. coli cell lysates with overexpressed CPSA plasmids. Each E. coli lysate was evaluated for the presence of (1) BP, (2) the expected CPSA intermediate, and (3) the subsequent intermediate, with the exception of pBAS11. Cells expressing pBAS11, lacking the C4 epimerase, wbpP, did not produce the CPSA BPP-linked trisaccharide. Only BPP-linked pyruvylated disaccharide (BPP-pyrDi) could be detected in cell expressing pBAS11. pBAS15 does include wbpP, and BPP-linked trisaccharide was detected in these lysates. Plasmids are described in Table 4.

FIG. 12 is a whole-cell dot blot of E. coli DH5α expressing CPSA plasmids and showing that E. coli DH5a expressing pBAS16 and pBAS17 reacted with anti-CPSA antiserum. The plasmids employed for this dot blot are described in Table 4. A positive signal for CPSA was only observed with cells expressing the pBAS16 or the pBAS17 plasmid.

FIG. 13 graphs the LC-MS analysis of BPP-linked CPSA tetrasaccharide in E. coli cell lysates. Normalized cell lysates expressing pBAS16 (−wzx, −wzy; left panel) or pBAS17 (+wzx, +wzy; right panel) were evaluated for the presence of BP and BPP-linked CPSA tetrasaccharide using ESI-LC-MS with SIM. BPP-tetrasaccharide was still present in pBAS17 cell lysates but appeared to be much less abundant compared to cells expressing pBAS16.

FIGS. 14A and 14B are schematic diagrams of an exemplary pathway for producing CPSA in bacteria (FIG. 14A) and a representative arrangement of various coding sequences in the expression cassettes and vectors of the presently disclosed subject matter (FIG. 14B).

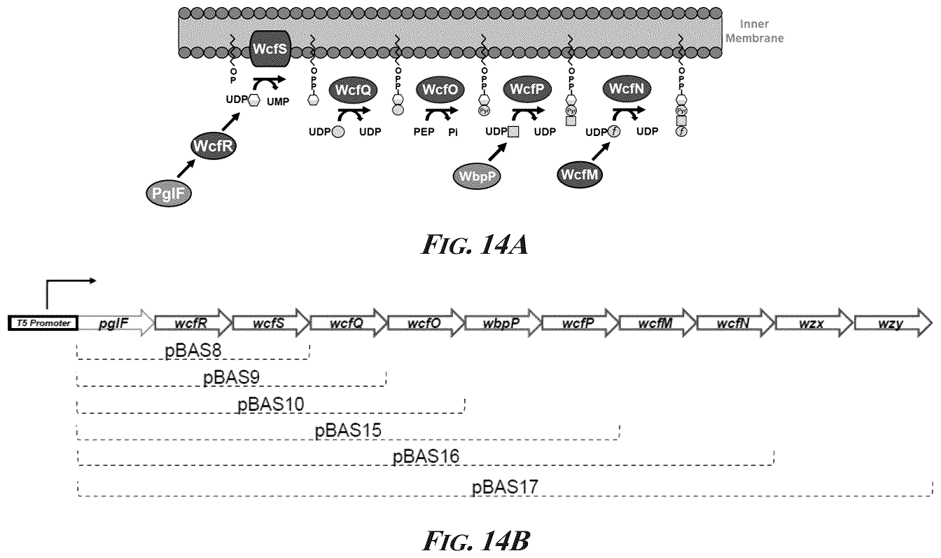

FIG. 15 is a map of plasmid pBAS17, which includes the coding sequences shown in FIG. 14B.

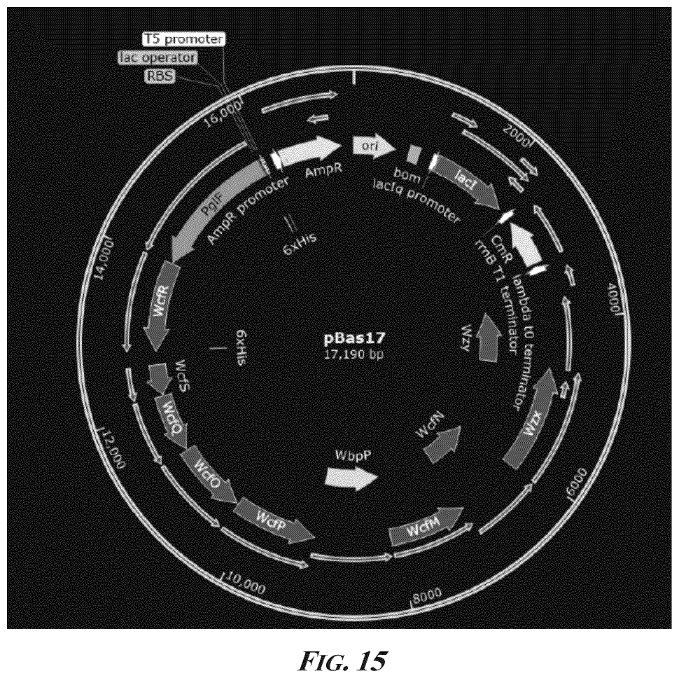

### DETAILED DESCRIPTION

The presently disclosed subject matter now will be described more fully hereinafter in the following detailed description of the presently disclosed subject matter, in which some, but not all embodiments of the presently disclosed subject matter are described. Indeed, the presently disclosed subject matter may be embodied in many different forms and should not be construed as limited to the embodiments set forth herein; rather, these embodiments are provided so that this disclosure will satisfy applicable legal requirements.

### I. Definitions

Unless otherwise defined, all technical and scientific terms used herein have the same meaning as commonly understood by one of ordinary skill in the art to which the presently described subject matter belongs.

Throughout the specification and claims, a given chemical formula or name shall encompass all active optical and stereoisomers, as well as racemic mixtures where such isomers and mixtures exist.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “a protein” refers to one or more proteins, including a plurality of the same protein. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments +20%, in some embodiments +10%, in some embodiments +5%, in some embodiments +1%, in some embodiments +0.5%, and in some embodiments +0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “adjuvant” refers to a substance that elicits an enhanced immune response when used in combination with a specific antigen.

As used herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole. With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil.

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

As used herein, the terms “condition”, “disease condition”, “disease”, “disease state”, and “disorder” refer to physiological states in which diseased cells or cells of interest can be targeted with the compositions of the presently disclosed subject matter.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

In some embodiments, the terms “fragment”, “segment”, or “subsequence” as used herein refers to a portion of an amino acid sequence, comprising at least one amino acid, or a portion of a nucleic acid sequence comprising at least one nucleotide. Thus, in some embodiments, the terms “fragment”, “segment”, and “subsequence” are used interchangeably herein. In some embodiments, the term “fragment” refers to a compound (e.g., a small molecule compound, such as a small molecule comprising a purine scaffold) that can react with a reactive amino acid residue (e.g., a reactive cysteine) to form an adduct comprising a modified amino acid residue. Thus, in some embodiments, the terms “fragment” and “ligand” are used interchangeably. In some embodiments, the term “fragment” refers to that portion of a ligand that remains covalently attached to the reactive amino acid residue.

As used herein, a “ligand” is a compound (e.g., a purine-based compound) that specifically binds to a target compound or molecule, such as a reactive nucleophilic amino acid residue in a protein. In some embodiments, the ligand can bind to the target covalently. A ligand “specifically binds to” or “is specifically reactive with” a compound (e.g., a reactive amino acid residue) when the ligand functions in a binding reaction which is determinative of the presence of the compound in a sample of heterogeneous compounds.

As used herein, a “functional” biological molecule is a biological molecule in a form in which it exhibits a property by which it can be characterized. A functional enzyme, for example, is one that exhibits the characteristic catalytic activity by which the enzyme can be characterized.

As used herein, the term “linker” refers to a molecule that joins two other molecules either covalently or noncovalently, such as but not limited to through ionic or hydrogen bonds or van der Waals interactions.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

As used herein, the term “mass spectrometry” (MS) refers to a technique for the identification and/or quantitation of molecules in a sample. MS includes ionizing the molecules in a sample, forming charged molecules; separating the charged molecules according to their mass-to-charge ratio; and detecting the charged molecules. MS allows for both the qualitative and quantitative detection of molecules in a sample. The molecules can be ionized and detected by any suitable means known to one of skill in the art. Some examples of mass spectrometry are “tandem mass spectrometry” or “MS/MS,” which are the techniques wherein multiple rounds of mass spectrometry occur, either simultaneously using more than one mass analyzer or sequentially using a single mass analyzer. The term “mass spectrometry” can refer to the application of mass spectrometry to protein analysis. In some embodiments, electrospray ionization (ESI) and matrix-assisted laser desorption/ionization (MALDI) can be used in this context. In some embodiments, intact protein molecules can be ionized by the above techniques, and then introduced to a mass analyzer. Alternatively, protein molecules can be broken down into smaller peptides, for example, by enzymatic digestion by a protease, such as trypsin. Subsequently, the peptides are introduced into the mass spectrometer and identified by peptide mass fingerprinting or tandem mass spectrometry.

As used herein, the term “mass spectrometer” is used to refer an apparatus for performing mass spectrometry that includes a component for ionizing molecules and detecting charged molecules. Various types of mass spectrometers can be employed in the methods of the presently disclosed subject matter. For example, whole protein mass spectroscopy analysis can be conducted using time-of-flight (TOF) or Fourier transform ion cyclotron resonance (FT-ICR) instruments. For peptide mass analysis, MALDI time-of-flight instruments can be employed, as they permit the acquisition of peptide mass fingerprints (PMFs) at high pace. Multiple stage quadrupole-time-of-flight and the quadrupole ion trap instruments can also be used.

As used herein, the term “Western blot,” which can be also referred to as “immunoblot”, and related terms refer to an analytical technique used to detect specific proteins in a sample. The technique uses gel electrophoresis to separate the proteins, which are then transferred from the gel to a membrane (typically nitrocellulose or PVDF) and stained, in membrane, with antibodies specific to the target protein.

The expression “stable isotope labeling by amino acids in cell culture” (SILAC) is used herein to refer to an approach for incorporation of a label into proteins for mass spectrometry (MS)-based quantitative proteomics. SILAC comprises metabolic incorporation of a given “light” or “heavy” form of the amino acid into the proteins. For example, SILAC comprises the incorporation of amino acids with substituted stable isotopic nuclei (e.g. deuterium, ¹³C, ¹⁵N). In an illustrative SILAC experiment, two cell populations are grown in culture media that are identical, except that one of them contains a “light” and the other a “heavy” form of a particular amino acid (for example, ¹²C and ¹³C labeled L-lysine, respectively). When the labeled analog of an amino acid is supplied to cells in culture instead of the natural amino acid, it is incorporated into all newly synthesized proteins. After a number of cell divisions, each instance of the amino acid is replaced by its isotope-labeled analog. Since there is little chemical difference between the labeled amino acid and the natural amino acid isotopes, the cells behave substantially similar to the control cell population grown in the presence of a normal amino acid.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains proteins, cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### II. Exemplary Compositions and Methods of Use Thereof

### II.A. Introduction

In describing the presently disclosed subject matter, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

A goal of the presently disclosed subject matter was to devise a recombinant system for the overproduction of capsular polysaccharide A (CPSA) from Bacteroides fragilis NTCT 9343. CPSA belongs to a unique class of zwitterionic polysaccharides that possess a distinct charge motif of alternating positive and negative charges. In contrast to neutral carbohydrates, zwitterionic polysaccharides elicit a potent, T-cell dependent immune response.5-6 These distinctive and potent immunomodulatory properties of CPSA and other zwitterionic polysaccharides are modulated by both positively and negatively charged substituents, as well as their helical structure (see Tzianabos et al., 2000; Wang et al., 2000; Choi et al., 2002; Tzianabos et al., 2001). Akin to an immune response elicited by a proteinaceous antigen, CPSA is processed by antigen presenting cells and presented to CD4+ T-cells via the major histocompatibility complex II (MHCII). See e.g., Hergetet al. (2008) Statistical analysis of the Bacterial Carbohydrate Structure Data Base (BCSDB): characteristics and diversity of bacterial carbohydrates in comparison with mammalian glycans. BMC Struct Biol August 11:8:35; Surana & Kasper (2012) The yin yang of bacterial polysaccharides: lessons learned from B. fragilis PSA. Immunol Rev 245(1):13-26; Porter & Martens. (2017) The Critical

Roles of Polysaccharides in Gut Microbial Ecology and Physiology. Annu Rev Microbiol 71:349-369. As such, CPSA has been employed in the development of completely carbohydrate-based vaccines (Troy & Kasper (2010) Beneficial effects of Bacteroides fragilis polysaccharides on the immune system. Front Biosci (Landmark Ed) 15:25-34). Paradoxically, CPSA also activates innate immune responses by stimulating the production of anti-inflammatory cytokines, such as IL-10 (Eribo et al. (2021) The intestinal commensal, Bacteroides fragilis, modulates host responses to viral infection and therapy: Lessons for exploration during Mycobacterium tuberculosis infection. Infect Immun 90(1):e0032121; Tolonen et al. (2022) Synthetic glycans control gut microbiome structure and mitigate colitis in mice. Nature Communications 13(1):1244). The activation of anti-inflammatory cytokines has advanced CPSA as a potential therapeutic for inflammation-associated diseases such as multiple sclerosis, ulcerative colitis, viral encephalitis, and irritable bowel syndrome (Tolonen et al. (2022) Synthetic glycans control gut microbiome structure and mitigate colitis in mice. Nature Communications 13 (1): 1244; Zheng et al. (2020) Capsular Polysaccharide From Bacteroides fragilis Protects Against Ulcerative Colitis in an Undegraded Form. Frontiers in Pharmacology 11:570476; Ramakrishna et al. (2019) Bacteroides fragilis polysaccharide A induces IL-10 secreting B and T cells that prevent viral encephalitis. Nature Communications 10(1):2153; Nothaft et al. (2016) Engineering the Campylobacter jejuni N-glycan to create an effective chicken vaccine. Scientific Reports 6(1):26511.

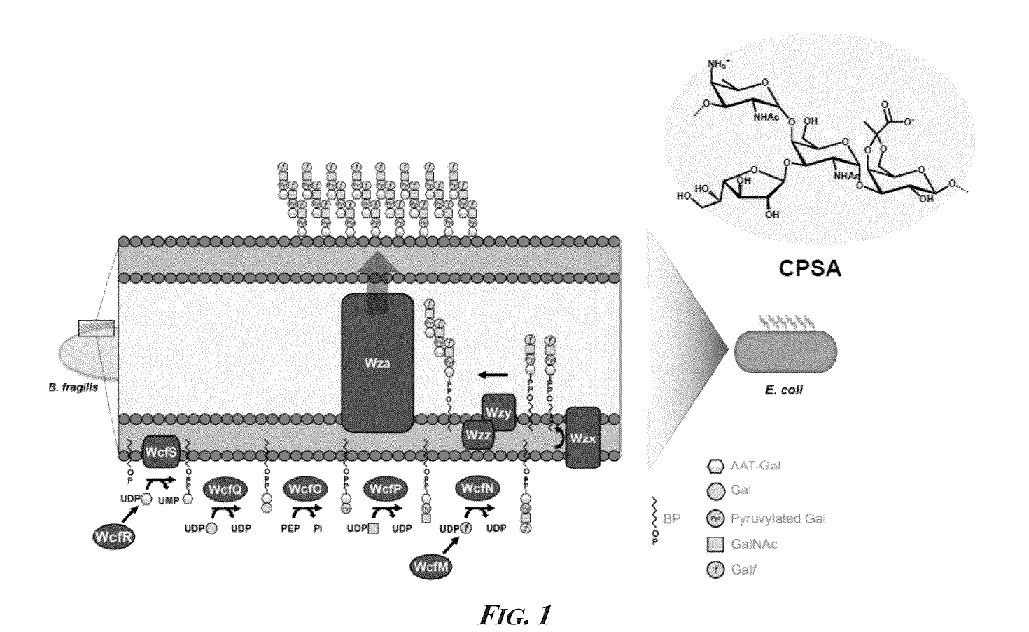

CPSA, a zwitterionic polymer, is composed of a repeating tetrasaccharide consisting of [→3)-β-d-pyrGalp-(1→3)-α-d-AATGalp-(1→4)[β-d-Galf-(1→3)]-α-d-GalpNAc-(1→], where AAT-Gal is 2-acetamido-4-amino-2,4,6-trideoxygalactopyranose, and pyrGal is 4,6-O-pyruvate-galactopyranose (FIG. 1). See Baumann et al., 1992. It is assembled on a common lipid anchor, bactoprenyl phosphate (BP; also known as undecaprenyl phosphate), at the cytoplasmic face of the inner membrane. Biosynthesis begins with the addition phospho-AATGal to BP by the initiating phospho-glycosyltransferase WcfS to produce BPP-AATGal (FIG. 1). See Mostafavi & Troutman, 2013. The remaining sugars, galactose (Gal), N-acetylgalactosamine (GalNAc), and galactofuranose (Galf) are sequentially appended to BPP-AATGal by the glycosyltransferases WcfQ, WcfP, and WcfN, respectively (Sharma et al., 2017). The pyruvyltransferase, WcfO, pyruvylates galactose after the formation of BPP-AATGal-Gal, and before the additional of GalNAc (Sharma et al., 2017). WcfM is a UDP-galactopyranose mutase that produces UDP-galactofuranose, the final sugar in CPSA repeat unit biosynthesis.

As disclosed herein, a recombinant system for CPSA production in E. coli was developed by implementing a stepwise method and strategic incorporation of unrelated, robust glycan modification enzymes. In doing so, the production of each lipid linked intermediate was achieved, and CPSA polymers of biologically relevant length were produced. Ultimately, an effective approach was modeled to evaluate recombinant polysaccharide production, which may be applied to other glycan biosynthesis systems that are not confined within a single operon. These results could aid in identification of potential targets to increase recombinant polymer quantity and increase availability of biologically relevant glycans.

### II.B. Compositions: Nucleic Acids

In some embodiments, the presently disclosed subject matter provides nucleic acids that encode one or more biosynthetic enzymes that are involved in polysaccharide production in bacteria. By way of example and not limitation, the presently disclosed subject matter provides nucleic acids that encode one or more polypeptides that can be employed to produce a capsular polysaccharide A (CPSA) in a bacterium. CPSA can be produced via the actions of various genes from Bacteroides fragilis (optionally Bacteroides fragilis NTCT 9343), and in some embodiments the presently disclosed nucleic acid molecules employ one or more of the genes found in Bacteroides fragilis. These genes include the wcfR aminotransferase, the wcfS phospho-glycosyltransferase, the wcfQ glycosyltransferase, the wcfO pyruvyltransferase, the wcfP glycosyltransferase, the wcfM UDP-galactopyranose mutase, and the wcfN glycosyltransferase gene products, the nucleic acid and amino acid sequences of which are known. Exemplary such sequences include SEQ ID NOs: 79 (nucleic acid) and 80 (amino acid for wcfR, SEQ ID NOs: 81 and 82 for wcfS, SEQ ID NOs: 83 and 84 for wcfQ, SEQ ID NOs: 85 and 86 for wcfO, SEQ ID NOs: 87 and 88 for wcfP, SEQ ID NOs: 89 and 90 for wcfM, and SEQ ID NOs: 91 and 92 for wcfN.

In some embodiments, coding sequences for one or more of these gene products is provided to a bacterium, in some embodiments as part of an expression cassette. In some embodiments, at least one of the coding sequences is derived from an organism other than the species of the bacterium in which the coding sequences are introduced (i.e., in some embodiments, at least one coding sequence is heterologous to the bacterium.

As disclosed herein, additional gene products can also be employed to increase the efficiency and/or yield of the polysaccharide of interest. Such additional gene products can include, but are not limited to a pglF dehydrogenase gene product (optionally, a Campylobacter jejuni pglF gene product), a wbpP UDP-N-acetyl-d-glucosamine C4 epimerase gene product (optionally, a Vibrio vulnificus wbpP gene product), a wza polysaccharide export protein gene product (optionally an E. coli gene product, further optionally encoded by SEQ ID NO: 93 and/or having the amino acid sequence set forth in SEQ ID NO: 94), a wzx fippase gene product (optionally a Bacteroides fragilis gene product, further optionally encoded by SEQ ID NO: 95 and/or having the amino acid sequence set forth in SEQ ID NO: 96), and/or a wzy polymerase gene product (optionally a Bacteroides fragilis gene product, further optionally encoded by SEQ ID NO: 97 and/or having the amino acid sequence set forth in SEQ ID NO: 98).

As such, in some embodiments the presently disclosed subject matter provides expression cassettes comprising a plurality of coding sequences derived from at least two different bacterial species, wherein the plurality of coding sequences are selected from the group consisting of a pglF dehydrogenase gene coding sequence, a wbpP UDP-N-acetyl-d-glucosamine C4 epimerase gene coding sequence, a wcfR aminotransferase gene coding sequence, a wcfS phospho-glycosyltransferase gene coding sequence, a wcfQ glycosyltransferase gene coding sequence, a wcfO pyruvyltransferase gene coding sequence, a wcfP glycosyltransferase gene coding sequence, a wcfM UDP-galactopyranose mutase gene coding sequence, a wcfN glycosyltransferase gene coding sequence, a wza polysaccharide export protein gene coding sequence, a wzx fippase gene coding sequence, a wzy polymerase gene coding sequence, and a wzz gene coding sequence.

It is understood that within the scope of the presently disclosed subject matter are expression cassettes that comprise fewer than the full complement of coding sequences listed above. By way of example and not limitation, in some embodiments the expression cassette comprises a combination of coding sequences selected from the group consisting of:
pglF, wcfR, and wcfS;
pglF, wcfR, wcfS, and wcfQ;
pglF, wcfR, wcfS, wcfQ, and wcfO;
pglF, wcfR, wcfS, wcfQ, wcfO, wbpP, and wcfP;
pglF, wcfR, wcfS, wcfQ, wcfO, wbpP, wcfP, wcfM, and wcfN; and
pglF, wcfR, wcfS, wcfQ, wcfO, wbpP, wcfP, wcfM, wcfN, wzx, and wzy.

In order to express the plurality of coding sequences, in some embodiments the expression cassette of the presently disclosed subject matter is operatively linked to a promoter such that transcription from the promoter results in the production of a polycistronic mRNA that is translatable to produce polypeptides encoded by the coding sequences. In some embodiments, the polycistronic mRNA molecule comprises a ribosome-binding site (RBS) operatively linked to a translation initiation codon of one or more of, optionally each of, the plurality of coding sequences, wherein at least one RBS is located 3′ to a translation termination codon of a first coding sequence and 5′ to a translation initiation codon of a second coding sequence, wherein the first coding sequence and the second coding sequence are adjacent to each other on the polycistronic mRNA molecule, the first coding sequence being present 5′ to the second coding sequence. In some embodiments, the RBS comprises the nucleic acid sequence 5′-AGGAGG′-3′. In some embodiments, the RBS is attached to a linker sequence to form an RBS domain, wherein the RBS domain is just 5′ of the translation initiator sequence of a coding sequence present in the expression cassette. In some embodiments the RBS/linker sequence comprises, consists essentially of, or consists of the nucleotide sequence 5′-AGGAGGATATACAT (SEQ ID NO: 100), wherein nucleotides 1-6 are the RBS and nucleotides 7-14 are the linker sequence.

In some embodiments, the polycistronic mRNA includes a plurality of RBSs, one operatively linked to each coding sequence. The RBSs that are present in the expression cassette (and hence the mRNA transcribed therefrom), can be the same or different. In some embodiments, the RBS cassette of SEQ ID NO: 100 is present just upstream of the initiator codon of each of the coding sequences present in the expression cassette.

In some embodiments, the expression cassette is transcribed in a bacterium in order to express the one or more coding sequences present thereon. In some embodiments, the expression cassette is transcribed to produce a polycistronic mRNA. In some embodiments, the polycistronic mRNA molecule is operably linked to a promoter that is active in the bacterium, optionally wherein the promoter is an inducible promoter, optionally an isopropyl-β-d-thiogalactopyranoside (IPTG)-inducible promoter, further optionally wherein the promoter is a T5 promoter. An exemplary T5 promoter comprises the nucleotide sequence presented in SEQ ID NO: 99.

As disclosed herein, the expression cassettes of the presently disclosed subject matter can encode some or all of the proteins required to produce a capsular polysaccharide A (CPSA) in a bacterium. For example, if CPSA production is desired, the expression cassette can provide a full complement of genes for CPSA production, which in some embodiments can be each of pglF, wcfR, wcfS, wcfQ, wcfO, wbpP, wcfP, wcfM, wcfN, wzx, and wzy. Expressing each of—pglF, wcfR, wcfS, wcfQ, wcfO, wbpP, wcfP, wcfM, wcfN, wzx, and wzy in a bacterium can result in the bacterium producing CPSA. However, as shown in FIG. 1, an expression cassette that lacks a wcfN coding sequence would be expected to produce a trisaccharide of AT-Gal/pyruvylated Gal/GalNAc, whereas an expression cassette that lacks a wcfN coding sequence and a wcfP coding sequence would be expected to produce a disaccharide of AT-Gal/pyruvylated Gal. As such, the coding sequences that are present in the expression cassette can determine which polysaccharides are produced.

In some embodiments, an expression cassette of the presently disclosed subject matter is present in a vector, optionally an expression vector. Methods for generating vectors that comprise recombinant nucleic acids are known. See e.g., Sambrook & Russell (2001) Molecular Cloning: a Laboratory Manual, 3rd ed. Cold Spring Harbor Laboratory Press, Cold Spring Harbor, New York, United States of America.

### II.C. Recombinant Bacteria

In some embodiments, the presently disclosed subject matter relates to bacteria comprising the expression cassettes disclosed herein. As such, in some embodiments the presently disclosed subject matter relates to bacteria that have been transformed to carry an expression cassette comprising a plurality of coding sequences, the plurality of coding sequences comprising nucleotide sequences that encode two or more of a pglF dehydrogenase gene product, a wbpP UDP-GlcNAc C4 epimerase gene product, a wcfR aminotransferase gene product, a wcfS phospho-glycosyltransferase gene product, a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, wherein at least one of the plurality of coding sequences is heterologous to the bacterium.

Any bacterium for which production of recombinant polysaccharides would be of interest can be employed in the compositions and methods of the presently disclosed subject matter. In some embodiments, the bacterium is an E. coli bacterium.

The bacteria of the presently disclosed subject matter carry at least one gene that is heterologous to (i.e., derived from a bacterium of a different strain, species, or genus) than the bacterium itself. As such, the bacteria are designed to carry at least one coding sequence that is not naturally found in that bacterium. Thus, in some embodiments the bacterium of the presently disclosed subject matter is an E. coli bacterium, and at least one of the pglF gene product, the wbpP gene product, the wcfR gene product, the wcfS gene product, the wcfQ gene product, the wcfO gene product, the wcfP gene product, the wcfM gene product, the wcfN gene product, the wza gene product, the wzx flippase gene product, the wzy polymerase gene product, and/or the wzz gene product is not naturally occurring in the E. coli bacterium. By way of example and not limitation, in some embodiments, the pglF gene product is a Campylobacter jejuni gene product, the wbpP gene product is a Vibrio vulnificus gene product, the wcfR gene product is a Bacteroides fragilis gene product, the wcfS gene product is a Bacteroides fragilis gene product, the wcfQ gene product is a Bacteroides fragilis gene product, the wcfO gene product is a Bacteroides fragilis gene product, the wcfP gene product is a Bacteroides fragilis gene product, the wcfM gene product is a Bacteroides fragilis gene product, the wcfN gene product is a Bacteroides fragilis gene product, the wza gene product is an Escherichia coli gene product, the wzx flippase gene product is a Bacteroides fragilis gene product, the wzy polymerase gene product is a Bacteroides fragilis gene product, and/or the wzz gene product is a Bacteroides fragilis gene product.

In some embodiments, the plurality of coding sequences are transcribed in the bacterium to produce a polycistronic messenger RNA (mRNA) molecule in the bacterium. In some embodiments, the polycistronic mRNA molecule comprises a ribosome-binding site (RBS) operatively linked to a translation initiation codon of each of at least two of the plurality of coding sequences present therein. In some embodiments, the polycistronic mRNA molecule comprises a plurality of ribosome-binding sites (RBSs), with an RBS present 5′ to each of at least two, optionally 5′ to each, translation initiation codon present in the polycistronic mRNA, wherein at least one RBS is located 3′ to a translation termination codon of a first coding sequence and 5′ to a translation initiation codon of a second coding sequence present on the polycistronic mRNA molecule.

In some embodiments, translation of the polycistronic mRNA molecule in the bacterium results in production of a polysaccharide in the bacterium, optionally wherein the polysaccharide is present in a lipid bilayer of the bacterium, further optionally wherein the lipid bilayer is part of the bacterium's cell wall. In some embodiments, the polysaccharide is a CPSA, optionally an aggregate of two or more CPSA tetrasaccharide monomers.

It is noted that the various biological activities disclosed herein can be provided recombinantly using the nucleic acids and expression cassettes of the presently disclosed subject matter. However, it is also understood that for those biological activities for which the recombinant bacterium is modified to produce but that the recombinant bacteria already possesses such an activity, the nucleic acids of the presently disclosed subject matter can optionally exclude the relevant coding sequences and instead rely on the endogenous expression of those activities by the bacterium. As such, for those activities that the bacterium would be expected to express in the absence of the compositions of the presently disclosed subject matter, it is not required that such an activity would need to be provided.

Thus, for example, if the bacterium already expresses a wza gene product, a wzx flippase gene product, a wzy polymerase, and/or a wzz gene product, the biological activity can be provided endogenously.

### II.C. Methods of Producing Polysaccharides Using the Nucleic Acids of the Presently Disclosed Subject Matter

In some embodiments, the presently disclosed subject matter employs the nucleic acids, optionally the expression cassettes disclosed herein, in methods for producing polysaccharides in bacteris. In some embodiments, the methods comprise expressing in the bacterium a plurality of coding sequences, the plurality of coding sequences comprising nucleotide sequences that encode a pglF dehydrogenase gene product, a wbpP UDP-N-acetyl-d-glucosamine C4 epimerase gene product, a wcfR aminotransferase gene product, and a wcfS phospho-glycosyltransferase gene product, and optionally further comprise nucleotide sequences encoding one or more of a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, and further wherein at least one of the coding sequences is heterologous to the bacterium. In some embodiments, the polysaccharide is a capsular polysaccharide A (CPSA) and the bacterium expresses each of the PglF gene product, the wbpP gene product, the WcfR gene product, the WcfS gene product, the WcfQ gene product, the WcfO gene product, the WcfP gene product, the WcfM gene product, the WcfN gene product, the wzz gene product, the wzx fippase gene product, and the wzy polymerase gene product, at least one of which is heterologous to the bacterium. In some embodiments, the bacterium is an Escherichia coli bacterium.

In some embodiments, the pglF gene product is a Campylobacter jejuni gene product; the wbpP gene product is a Vibrio vulnificus gene product; the wcfR gene product is a Bacteroides fragilis gene product; the wcfS gene product is a Bacteroides fragilis gene product; the wcfQ gene product is a Bacteroides fragilis gene product; the wcfO gene product is a Bacteroides fragilis gene product; the wcfP gene product is a Bacteroides fragilis gene product; the wcfM gene product is a Bacteroides fragilis gene product; the wcfN gene product is a Bacteroides fragilis gene product; the wza gene product is an Escherichia coli gene product; the wzx fippase gene product is a Bacteroides fragilis gene product; the wzy polymerase gene product is a Bacteroides fragilis gene product; and/or the wzz gene product is a Bacteroides fragilis gene product.

In some embodiments, action of the coding sequences disclosed herein in the bacterium results in the presence of the polysaccharide is present in a lipid bilayer of the bacterium, optionally wherein the lipid bilayer is part of the bacterium's cell wall. In some embodiments, the bacterium does not express a functional waal O-antigen ligase gene product.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the EXAMPLES

General. Polyclonal anti-CPSA antibodies were generously provided by Dr. Laurie Comstock. All bacterial cultures were grown in Miller Lysogeny Broth unless otherwise specified. E. coli mutants and pQE-80L plasmid were generously provided by Dr. Matthew Jorgensen of the University of Arkansas Medical Center. LC-MS analysis was performed on an Agilent 1260 Infinity II equipped with a quaternary pump and MSD. All mobile phases were prepared from LC-MS grade reagents.

Construction of CPSA plasmids. CPSA plasmids were constructed using the primers listed in Table 1. Briefly, pQE80L vector was isolated from E. coli DH5a cells. The vector was digested with restriction enzymes BamHI and HindII and purified with gel electrophoresis. Gene inserts with amplified with Phusion DNA polymerase (New England Biolabs). Amplicons were purified using the WIZARD® SV Gel and PCR Clean-Up System (Promega). Purified, digested vector and inserts were incubated with NEBuilder HiFi DNA Assembly Mix (New England Biolabs) according to the manufacturer's instructions and incubated at 50° C., or overnight for constructs with more than 4 fragments. E. coli DH5α cells were then chemically transformed with 5 μL of assembly reaction mixture and plated on selective media (LB/Carb100). Successful transformants were confirmed by colony PCR.

An artificial SacI site was incorporated into pBAS8 immediately following wcfS. For construction of pBAS9-19, SacI digested pBAS8 was used as the vector backbone. For construction of pBAS16, wcfO and wcfQ were amplified as a single insert using pBAS10 as a template. For construction of pBAS 17-19, six genes (wcfOQP_{Bf}, wbpP_{Vv}, wcfMN) were amplified as a single fragment using pBAS16 as a template.

TABLE 1
Primers Employed to Recreate CPSA Biosynthesis in E. coli

| | Genes | |
|---|---|---|
| Plasmid | (Source) | Primers* |
| pBAS1 | pglF_{Cj} | CATCACCATCACCATCACGACATGATTTTTTATAAAAGCAAAAGATT |
| | (V. vulnificus | AGC (SEQ ID NO: 1) |
| | M06-24) | GGATTTGTTGGTATTTGGTCATATGTATATCCTCCTTTATACACCTTCT |
| | | TTATTGTGTTTAAATTC (SEQ ID NO: 2) |
| | wbpP_{Vv} | ATGACCAAATACCAACAAATCC (SEQ ID NO: 3) |
| | (V. vulnificus | GTTATTCTTTATTGATTGTCCCATATGTATATCCTCCTCTATTTTATAA |
| | M06-24) | ATCGCACATACC (SEQ ID NO: 4) |
| | CPSA operon | ATGGGACAATCAATAAAGAATAAC (SEQ ID NO: 5) |
| | (B. fragilis | GAGTCCAAGCTCAGCTAATTACTAACGAATGATGCTCCAAAATG |
| | ATCC 9343) | (SEQ ID NO: 6) |
| pBAS8 | pglF_{Cj} | GGATCGCATCACCATCACCATCACGgaATGATTTTTTATAAAAGCAAA |
| | (C. jejuni | AGATTAGC (SEQ ID NO: 7) |
| | ATCC) | GGTATTTTCATATGTATATCCTCCTTTATACACCTTCTTTATTGTG |
| | | (SEQ ID NO: 8) |
| | wcfR_{Bf} | AGGAGGATATACATATGAAAATACCTTTTTCACCACC (SEQ ID NO: 9) |
| | (B. fragilis | CAAATGTATATCCTCCTCTAGTGGTGGTGGTGGTGGTGTCGGTTTTCT |
| | ATCC 9343) | GCAATTACG (SEQ ID NO: 10) |
| | wcfS_{Bf} | CCACTAGAGGAGGATATACATTTGATCCGTTTTTTTGATATCG (SEQ |
| | (B. fragilis | ID NO: 11) |
| | ATCC 9343) | GAGTCCAAGCTCAGCTAATTAGAGCTCCTAACGAATGATGCTCC |
| | | (SEQ ID NO: 12) |
| pBAS9* | wcfQ_{Bf} | GCATCATTCGTTAGGAGCTCAGGAGGATATACATATGAAATTGGCTG |
| | (B. fragilis | TCATTTC (SEQ ID NO: 13) |
| | ATCC 9343) | CAAGCTCAGCTAATTAGAGCTTCACTTGTCGTCATCGTCTTTGTAGTC |
| | | ACGTACTGTTTTATAGATTATG (SEQ ID NO: 14) |
| pBAS10* | wcfQ_{Bf} | GCATCATTCGTTAGGAGCTCAGGAGGATATACATATGAAATTGGCTG |
| | (B. fragilis | TCATTTC (SEQ ID NO: 5) |
| | ATCC 9343) | CCTTCAACGTACTGTTTTATAGATT (SEQ ID NO: 16) |
| | wcfO_{Bf} | AATCTATAAAACAGTACGTTGAAGGAGGATATACATATGAGGAAGA |
| | (B. fragilis | TATTATTAACATATGG (SEQ ID NO: 17) |
| | ATCC 9343) | CAAGCTCAGCTAATTAGAGCTTCACTTGTCGTCATCGTCTTTGTAGTC |
| | | TGACATCATAAATTTATTACATATATTAATTAATC (SEQ ID NO: 18) |
| pBAS11* | wcfQ_{Bf} | GCATCATTCGTTAGGAGCTCAGGAGGATATACATATGAAATTGGCTG |
| | (B. fragilis | TCATTTC (SEQ ID NO: 19) |
| | ATCC 9343) | CCTTCAACGTACTGTTTTATAGATT (SEQ ID NO: 20) |
| | wcfO_{Bf} | AATCTATAAAACAGTACGTTGAAGGAGGATATACATATGAGGAAGA |
| | (B. fragilis | TATTATTAACATATGG (SEQ ID NO: 21) |
| | ATCC 9343) | CTCATATGTATATCCTCCTTCATGACATCATAAATTTATTACATATAT |
| | | TAATTAATC (SEQ ID NO: 22) |
| | wcfP_{Bf} | CATGAAGGAGGATATACATATGAGGGATGGAAAGCC (SEQ ID NO: |
| | (B. fragilis | 23) |
| | ATCC 9343) | CAAGCTCAGCTAATTAGAGCTTCACTTGTCGTCATCGTCTTTGTAGTC |
| | | TTTATGTAAAAGATTTAAATATATTCTTTC (SEQ ID NO: 24) |
| pBAS12* | wcfQ_{Bf} | GCATCATTCGTTAGGAGCTCAGGAGGATATACATATGAAATTGGCTG |
| | (B. fragilis | TCATTTC (SEQ ID NO: 25) |
| | ATCC 9343) | CCTTCAACGTACTGTTTTATAGATT (SEQ ID NO: 26) |
| | wcfO_{Bf} | AATCTATAAAACAGTACGTTGAAGGAGGATATACATATGAGGAAGA |
| | (B. fragilis | TATTATTAACATATGG (SEQ ID NO: 27) |
| | ATCC 9343) | CTCATATGTATATCCTCCTTCATGACATCATAAATTTATTACATATAT |
| | | TAATTAATC (SEQ ID NO: 28) |
| | wcfP_{Bf} | CATGAAGGAGGATATACATATGAGGGATGGAAAGCC (SEQ ID NO: |
| | (B. fragilis | 29) |
| | ATCC 9343) | CCTCCTTCATTTATGTAAAAGATTTAAATATATTCTTTCGAG (SEQ ID |
| | | NO: 30) |
| | wcfM_{Bf} | AAATCTTTTACATAAATGAAGGAGGATATACATATGAAAAAAAAAT |
| | (B. fragilis | ATGACTATCTAATTG (SEQ ID NO: 31) |
| | ATCC 9343) | CAACTACAGCAAATATCTTCATATGTATATCCTCCTTCATAAGTCACT |
| | | ATTTATAACTTTTTCCAC (SEQ ID NO: 32) |
| | wcfN_{Bf} | ATGAAGATATTTGCTGTAGTTG (SEQ ID NO: 33) |
| | (B. fragilis | CAAGCTCAGCTAATTAGAGCTTTACTTGTCGTCATCGTCTTTGTAGTC |
| | ATCC 9343) | ATAGGTAGCTCCATTTTTTTTACG (SEQ ID NO: 34) |
| pBAS15* | wcfQ_{Bf} | GCATCATTCGTTAGGAGCTCAGGAGGATATACATATGAAATTGGCTG |
| | (B. fragilis | TCATTTC (SEQ ID NO: 35) |
| | ATCC 9343) | CCTTCAACGTACTGTTTTATAGATT (SEQ ID NO: 36) |
| | wcfO_{Bf} | AATCTATAAAACAGTACGTTGAAGGAGGATATACATATGAGGAAGA |
| | (B. fragilis | TATTATTAACATATGG (SEQ ID NO: 37) |
| | ATCC 9343) | CTCATATGTATATCCTCCTTCATGACATCATAAATTTATTACATATAT |
| | | TAATTAATC (SEQ ID NO: 38) |
| | wcfP_{Bf} | CATGAAGGAGGATATACATATGAGGGATGGAAAGCC (SEQ ID NO: |
| | (B. fragilis | 39) |
| | ATCC 9343) | GTAAAAGATTTAAATATATTCTTTCGAG (SEQ ID NO: 40) |
| | wbpP_{Vv} | CTCGAAAGAATATATTTAAATCTTTTACATAAATGAAGGAGGATATA |
| | (V. vulnificus | CATATGACCAAATACGAAAAAATCC (SEQ ID NO: 41) |
| | M06-24) | CAAGCTCAGCTAATTAGAGCTTTATTTTTTATCATTTATAAAGCTTAT |
| | | ATACC (SEQ ID NO: 42) |
| pBAS16* | wcfQO_{Bf} | GCATCATTCGTTAGGAGCTCAGGAGGATATACATATGAAATTGGCTG |
| | (from pBAS10) | TCATTTC (SEQ ID NO: 43) |
| | | CTCATATGTATATCCTCCTTCATGACATCATAAATTTATTACATATAT |
| | | TAATTAATC (SEQ ID NO: 44) |
| | wcfP_{Bf} | CATGAAGGAGGATATACATATGAGGGATGGAAAGCC (SEQ ID NO: |
| | (B. fragilis | 45) |
| | ATCC 9343) | GTAAAAGATTTAAATATATTCTTTCGAG (SEQ ID NO: 46) |
| | wbpP_{Vv} | CTCGAAAGAATATATTTAAATCTTTTACATAAATGAAGGAGGATATA |
| | (V. vulnificus | CATATGACCAAATACGAAAAAATCC (SEQ ID NO: 47) |
| | M06-24) | CAAGCTCAGCTAATTAGAGCTTTATTTTTTATCATTTATAAAGCTTAT |
| | | ATACC (SEQ ID NO: 48) |
| | wcfM_{Bf} | GCTTTATAAATGATAAAAAATAAAGGAGGATATACATATGAAAAAA |
| | (B. fragilis | AAATATGACTATCTAATTG (SEQ ID NO: 49) |
| | ATCC 9343) | CAACTACAGCAAATATCTTCATATGTATATCCTCCTTCATAAGTCACT |
| | | ATTTATAACTTTTTCCAC (SEQ ID NO: 50) |
| | wcfN_{Bf} | ATGAAGATATTTGCTGTAGTTG (SEQ ID NO: 51) |
| | (B. fragilis | CAAGCTCAGCTAATTAGAGCTTTAATAGGTAGCTCCATTTTTTTTACG |
| | ATCC 9343) | (SEQ ID NO: 52) |
| pBAS17* | wcfQOP_wbpP_ | GCATCATTCGTTAGGAGCTCAGGAGGATATACATATGAAATTGGCTG |
| | wcfMN | TCATTTC (SEQ ID NO: 53) |
| | (from pBAS16) | CCTCCTTTAATAGGTAGCTCCATTTTTTTTACG (SEQ ID NO: 54) |
| | wxz_{Bf} | TGGAGCTACCTATTAAAGGAGGATATACATATGGGACAATCAATAAA |
| | (B. fragilis | GAATAAC (SEQ ID NO: 55) |
| | ATCC 9343) | CCTCCTTCATTGTTTAAATATTGAAAGTAATAATTTC (SEQ ID NO: 56) |
| | wzx_{Bf} | CAATATTTAAACAATGAAGGAGGATATACATATGACTAGTACTTCTT |
| | (B. fragilis | TCTTTATTATTAAG (SEQ ID NO: 57) |
| | ATCC 9343) | AAGCTCAGCTAATTAGAGCTTTAAAATTTGATTTTGGCATTAAAC |
| | | (SEQ ID NO: 58) |
| pBAS18* | wcfQOP_wbpP_ | GCATCATTCGTTAGGAGCTCAGGAGGATATACATATGAAATTGGCTG |
| | wcfMN | TCATTTC (SEQ ID NO: 59) |
| | (from pBAS16) | CCTCCTTTAATAGGTAGCTCCATTTTTTTTACG (SEQ ID NO: 60) |
| | wzx_{Bf} | TGGAGCTACCTATTAAAGGAGGATATACATATGGGACAATCAATAAA |
| | (B. fragilis | GAATAAC (SEQ ID NO: 61) |
| | ATCC 9343) | CAAGCTCAGCTAATTAGAGCTTCATTGTTTAAATATTGAAAGTAATA |
| | | ATTTC (SEQ ID NO: 62) |
| pBAS19* | wcfQOP_wbpP_ | GCATCATTCGTTAGGAGCTCAGGAGGATATACATATGAAATTGGCTG |
| | wcfMN | TCATTTC (SEQ ID NO: 63) |
| | (from pBAS16) | CCTCCTTTAATAGGTAGCTCCATTTTTTTTACG (SEQ ID NO: 64) |
| | wzy_{Bf} | TGGAGCTACCTATTAAAGGAGGATATACATATGGGACAATCAATAAA |
| | (B. fragilis | GAATAAC (SEQ ID NO: 65) |
| | ATCC 9343) | CAAGCTCAGCTAATTAGAGCTTCATTGTTTAAATATTGAAAGTAATA |
| | | ATTTC (SEQ ID NO: 66) |

*Plasmids constructed using SacI-digested pBAS8 as the vector backbone. See Materials and Methods.
**Sequences are presented in the 5′ to 3′ direction.

Extraction of BPP-linked CPSA intermediates. Cultures for glycolipid extraction were prepared by inoculating a single colony into 5 mL of LB/Carb¹⁰⁰ and 2% glucose. Cultures were grown overnight for 16 hour at 37° C., 220 rpm. Cell cultures were then diluted 1:1000 into 5 mL of LB/Carb¹⁰⁰ and grown at 37° C., 220 rpm until reaching an OD₆₀₀ of approximately 0.6 before induction with 0.1 mM IPTG. After overnight induction, liquid cultures were transferred to falcon tubes and centrifugated at 5,000×g for 15 minutes at 4° C.

The supernatant was discarded, and the cell pellet was resuspended in 10 mM PBS. The cell suspension was then transferred to a 15 mL glass centrifuge tube. To the cell suspension, 1 mL of chloroform and 2 mL of methanol were added to create a single-phase solution of water: chloroform: methanol (0.8:1:2.0). Each sample was vortexed and incubated at room temperature for 20 minutes to ensure cell lysis. Resulting insoluble materials were clarified from the cell lysate solution by centrifugation at 2,500×g for 20 minutes at room temperature. The supernatant was then transferred to a clean glass culture tube, frozen at −80° C., then dried under vacuum. Dried samples were resuspended in a solution of 1 mM ammonium hydroxide: n-propanol (1:1).

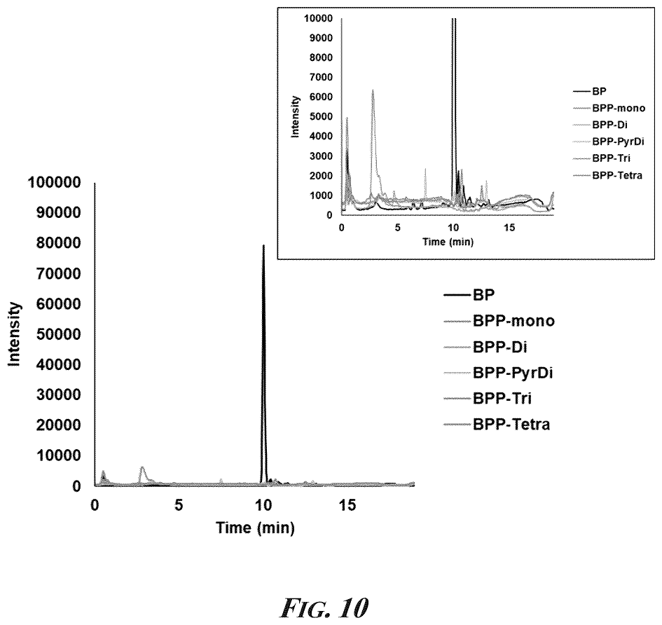

LC-MS analysis of BPP-linked CPSA intermediates. To evaluate the presence of CPSA intermediates in E. coli, 10 μL of cell extract was injected and separated using Waters XBridge BEH C18 column (5 μm, 4.6×100 mm, 300 Å). For LC-MS analysis, mobile phase A consisted of 0.1% ammonium hydroxide pH 11, and mobile phase B was n-propanol. BPP-linked intermediates were separated using a 5-75% gradient of mobile phase B over 12 minutes, then 75-95% B for 5 minutes, and an isocratic hold at 95% B for 5 minutes at 1mL/min for a total run time of 22 minutes. The column was equilibrated for 5 min at 5% B prior to the next injection. Intermediates were detected using selected ion monitoring (SIM) of the predicted [M-H]⁻ and/or [M-H]-⁻² ion of each intermediate. Total ion chromatograms were collected for each injection. Controls were prepared from parent strains expressing empty plasmids and evaluated for CPSA intermediates or isobaric compounds (FIG. 10).

Whole-cell dot blots. Cultures were prepared from single colonies, induced with 0.1 mM IPTG at OD600 nm, and grown overnight at 37° C. at 220 rpm. Whole-cell dot blots were prepared using 5 μL of liquid culture dried on nitrocellulose. After drying, the nitrocellulose was blocked with a 5% milk solution for 30 min. The nitrocellulose was then rinsed gently with deionized water (×3) and incubated at room temperature for 1 hour with 1 mL of an adsorbed CPSA antiserum diluted 1:30 in sterile 10 mM PBS. CPSA antiserum was provided by Dr. Laurie Comstock (see Comstock et al., 1999). After incubation with anti-CPSA, the nitrocellulose was washed twice for 5 min in 0.3% PBST, then placed in alkaline phosphatase conjugated anti-rabbit goat secondary antibody (1:10,000) for 1 hour at room temperature. The nitrocellulose was then washed 3× for 1 minute with 0.3% PBST and developed with NBT-BCIP.

SDS-PAGE and LPS staining. To evaluate LPS and CPS profiles of E. coli expressing either pBAS16 or pBAS17, overnight cultures were diluted 1:100 in LB and incubated at 37° C. with shaking until reaching an OD of ˜0.6. Cell cultures were then induced with 0.01 mM IPTG and incubated overnight at 37° C. Cell cultures were normalized to an OD₆₀₀ of 1.0, and a 0.5 mL volume of each sample was pelleted and resuspended in 100 μL of 1× SDS dye. Cell samples were then lysed at 95° C. for 10 min and cooled to room temperature before 20 μg of pronase (Sigma) was added. The samples were incubated at room temperature for 20 min prior to SDS-PAGE. A 5 μL sample of each lysate was added to individual wells and separated at 40 mA for 60 min. Gels were then stained with the PRO-Q™ Emerald 300 kit (Thermo Fisher Scientific) according to the manufacturer's instructions. Generation of E. coli mutants. E. coli mutants (Table 2) were either generously provided by the Jorgenson laboratory or prepared using the lambda red recombinase method of Datsenko & Wanner, 2000. Briefly, parent strains of E. coli were transformed with pKD46. Single colonies of transformants were used to inoculate 5 mL cultures containing LB/Carb¹⁰⁰ and incubated at 30° C. Cultures were induced with 100 mM L-arabinose and returned to the incubator for 1 hour at 30° C. Cells were then pelleted at 5,000 RCF at 4° C. and washed six times with ice-cold 10% glycerol. These cells were used immediately for transformation or stored as aliquots at −80° C. For transformation, approximately 1-2 μg of purified amplicon was added to cell suspensions. Cells were then electroporated at 2,500 V and immediately recovered in LB at 30° C. overnight. Recovered cells were plated on LB/Cam²⁰ to select for waaL mutants. Colony PCR was used to confirm the correct insertion of antibiotic resistance cassettes. Primers used to generate and confirm mutants are listed in Table 3. BAS13 was prepared by transforming DR35, a strain provided by the Young Laboratory, with pCP20 to remove the Kan resistance cassette (Ranjit & Young, 2016). To ensure that the Kan resistance cassette was successfully removed, cells were tested for sensitivity against Kan⁵⁰. To cure the cells of the pCP20 plasmid, cells were cultured on LB agar and incubated at 37° C. and colony purified twice more at 37° C. Plasmid curing was confirmed by testing cells for sensitivity to Carb¹⁰⁰.

TABLE 2
E. coli Strains Employed

| Strain | Genotype |
|---|---|
| DH5α | F- φ80lacZΔM15 Δ(lacZYA-argF)U169 recA1 endA1 |
| | hsdR17(rK−, mK+) phoA supE44 λ-thi-1 gyrA96 relA1 |
| MG1655 | K-12 F⁻ λ⁻ ilvG⁻ rfb-50 rph-1 |
| DR35 | Reference |
| BAS5 | MG1655 ΔwzxB::frt |
| BAS13 | MG1655 Δwza::frt |
| MAJ975 | MG1655 ΔwaaL::frt |
| BAS24 | MG1655 Δwza::frtΔwaaL::cam |

TABLE 3
Primers Employed for E. coli Mutants

| Strain | | | |
|---|---|---|---|
| Designation | Primers | Template | Check Primers |
| BAS5 | GAAAGGCTCTTTACGTTAGATGAGCTTAT | pKD4 | TGGCTGCTATTGGGC |
| | CAGATTAAAATTAATTGCATGACATTACA | | GAA (SEQ ID NO: 71) |
| | CGTCTTGAGCGAT (SEQ ID NO: 67) | | TCCACCGATATGATT |
| | GCACAAACGGCACCAAACAAACCAGAAC | | TCTTTTC (SEQ ID NO: |
| | CAACAATGATATAATCGTACATATGAATA | | 72) |
| | TCCTCCTTAGTTCC (SEQ ID NO: 68) | | |
| BAS24 | TCAACAGTCAAGCAGTTTTGGAAAAGTTA | pKD3 | TATCCCAATGGCATC |
| | TCATCATTATAAAGGTAAAACTGAATATC | | G (SEQ ID NO: 73) |
| | CTCCTTAGTTCC (SEQ ID NO: 69) | | ACCCTAATTCACGTA |
| | TTGTATAGATAAGAAGTGAGTTTTAACTC | | CTCC (SEQ ID NO: 74) |
| | ACTTCTTAAACTTGTTTATTCATTGTGTAG | | |
| | GCTGGAGC (SEQ ID NO: 70) | | |

Confocal Microscopy. Cultures of E. coli MG1655 pQE-80L and MG1655 pBAS17 were prepared from single colonies. Once cultures reached an OD600nm of 0.6, they were induced with 0.1 mM IPTG for 2 hours. Cells were pelleted at 5,000×g for 15 minutes at 4° C. and washed twice with 5 mL of 10 mM PBS. Cells were then resuspended 100 μL of 10 mM PBS. Primary anti-CPSA antiserum was added to cell suspensions at a final concentration of 1:25, to make a final cell suspension of 100 μL. Cells were incubated with CPSA-antiserum at room temperature with rocking for 30 minutes. Cells were then pelleted at 10,000×g for 5 minutes and washed twice with 1 mL of PBS. Cells were resuspended in a 1:2,500 dilution of FITC conjugated secondary antibody and incubated at room temperature with rocking for 30 minutes. Cells were again pelleted at 10,000×g for 5 minutes and washed once with 10 mM PBS. Cells were resuspended in 100 μL of 10 mM PBS and 10 μL were dropped onto 0.7% agar pads prepared on a clean glass slide. Once cells were dried, a cover slip was added. Confocal microscopy was performed by Dr. Shreya Goyal and Dr. James Grissom.

### Example 1

### Construction of a CPSA Expression Plasmid

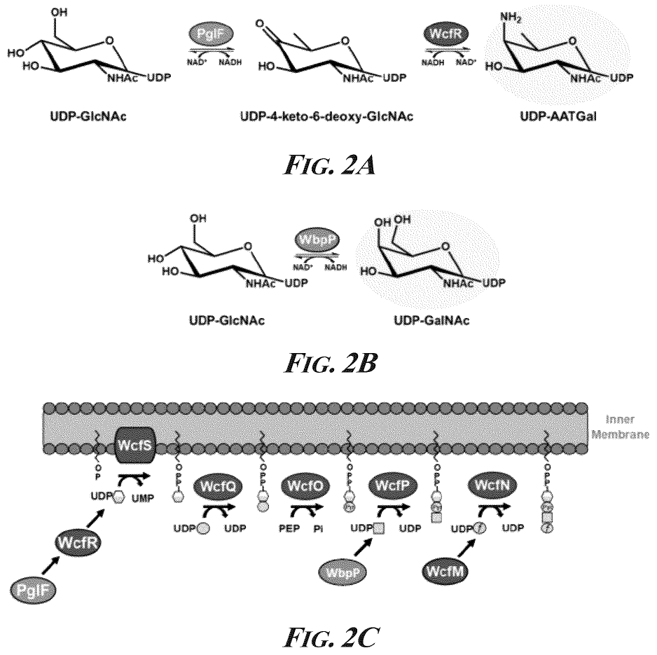

To produce CPSA in E. coli, we first sought to assemble the CPSA locus from B. fragilis into a plasmid. However, the CPSA locus does not contain all genes required for the assembly of UDP-AATGal and UDP-GalNAc, which are also not native to our E. coli host strains. In B. fragilis, UDP-AATGal is formed through the activity of a dehydrogenase that produces a 4-keto sugar, and an aminotransferase that adds an amine to the 4-position to produce a sugar with a 4-aminogalactose configuration. UDP-GalNAc could be readily formed through the epimerization of the E. coli abundant NDP-sugar UDP-GlcNAc. To ensure all NDP-sugars required for CPSA biosynthesis were available, Gibson assembly was used to incorporate pglF, a dehydrogenase from Campylobacter jejuni, which catalyzes dehydration of the 6-position of UDP-GlcNAc and oxidation of the 4-position to give UDP-2-acetamido-2,6-dideoxy-α-d-xylo-hexos-4-ulose, a precursor of UDP-AATGal (Olivier et al., 2006; Mostafavi & Troutman, 2013; Riegert et al., 2017). UDP-AATGal is formed from the 4-keto sugar by the aminotransferase WcfR, a gene product from the CPSA locus (FIG. 2A; see also Mostafavi & Troutman, 2013). We also incorporated wbpP from Vibrio vulnificus M0-624, a UDP-GlcNAc C4 epimerase that produces UDP-GalNAc (FIG. 2B; see also Park et al., 2006; Cunneen et al., 2013). Previous work from our laboratory and others have shown that PglF and WbpP are readily overproduced and functional after purification from E. coli (Schoenhofen et al., 2006; Reid et al., 2023).

Implementation of recombinant glycan production in E. coli has been demonstrated through the complete transfer of the biosynthesis operon into a plasmid for expression (Wacker et al., 2002; Ma & Wang, 2019). While this can be robust, it does not allow for evaluation of individual steps in the biosynthetic pathway, and bottlenecks in production can be challenging to delineate. To evaluate potential bottlenecks that may hinder CPSA biosynthesis in E. coli, we generated an incremental set of plasmids designed to produce each individual BPP-linked CPSA intermediate in a stepwise manner (FIG. 1, Table 4). It is important to note that each gene in Gibson assembled plasmids was preceded by a strong E. coli ribosomal binding site (RBS) often used for over production of gene products. Assembly of each plasmid was confirmed by PCR.

TABLE 4
Description of Plasmids used to Generate Individual
CPSA Intermediates in E. coli

| Plasmid | | |
|---|---|---|
| designation | Genes incorporated | Predicted CPSA Intermediate |
| pQE | [none] | BP |
| pBAS8 | pglF_{Cj}, wcfRS | BPP-AATGal |
| pBAS9 | pglF_{Cj}, wcfRSQ | BPP-AATGal-Gal |
| pBAS10 | pglF_{Cj}, wcfRSQO | BPP-AATGal-PyrGal |
| pBAS11 | pglF_{Cj}, wcfRSQOP | BPP-AATGal-PyrGal |
| pBAS12 | pglF_{Cj}, wcfRSQOPMN | BPP-AATGal-PyrGal |
| pBAS15 | pglF_{Cj}, wbpP_{Vv} | BPP-AATGal-PyrGal-GalNAc |
| | wcfRSQOP | |
| pBAS16 | pglF_{Cj}, wbpP_{Vv} | BPP-AATGal-PyrGal-GalNAc- |
| | wcfRSQOPMN | Galf |

### Example 2

### Stepwise Analysis of CPSA Repeat Unit Biosynthesis by ESI-LC-MS

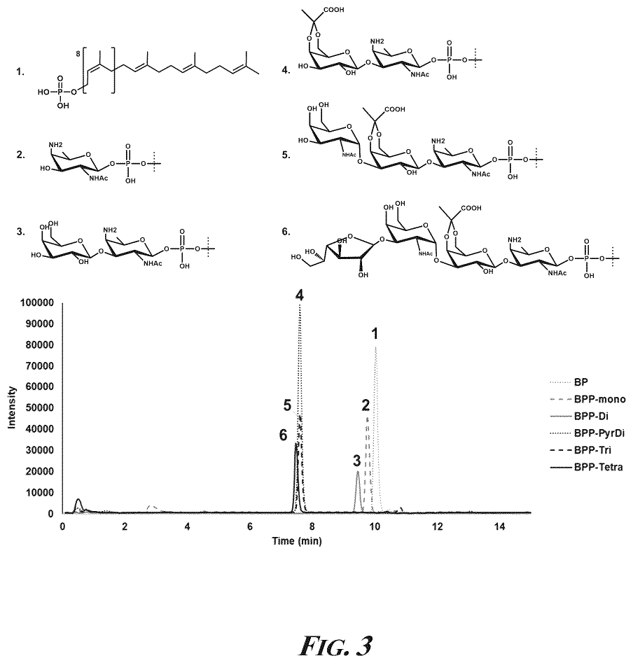

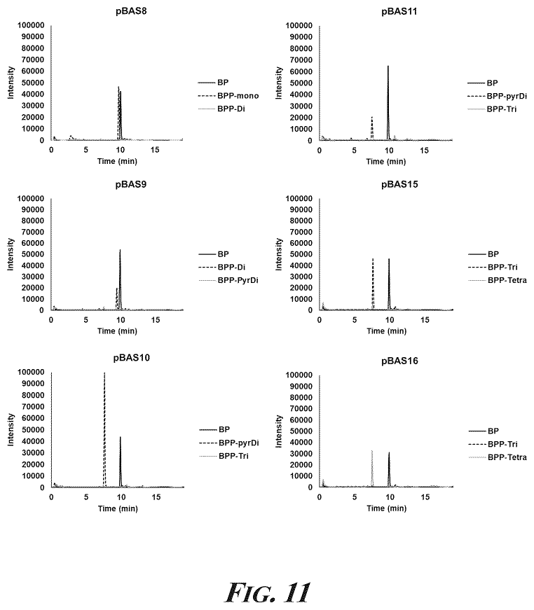

To investigate production of lipid-linked CPSA intermediates, we utilized an established ESI-LC-MS based method for tracking glycan intermediates in E. coli (Reid et al., 2021). Previously, we showed the precise order and function of enzymes involved in CPSA production in vitro, which enabled us to predict the m/z of BPP-linked CPSA intermediates based on which genes were expressed (Table 4; see Sharma et al., 2017). To ensure that each intermediate corresponded to the inclusion of the appropriate Wcf glycosyltransferase(s), we used selected ion monitoring (SIM) to analyze each cell lysate for the [M-H]⁻ and/or [M-H]²⁻ ion of (1) BP, (2) the expected BPP-linked intermediate, and (3) the subsequent predicted intermediate (FIG. 3). Spectra were collected from the total ion count (TIC) of each injection (FIG. 1). We chose to monitor for later intermediates in the pathway to confirm that CPSA intermediates were not produced without the incorporation of the appropriate genes. Intermediates only corresponding to what was predicted were identified in cell lysates (i.e., cells did not produce later intermediates until the appropriate genes were expressed). Additionally, we did not observe isobaric compounds with identical or similar retention times of CPSA intermediates in cells harboring an empty vector (FIG. 10). BPP-linked pyruvylated trisaccharide was not produced until wbpP_{Vv} was co-expressed with wcfP and preceding glycosyltransferases (FIG. 11), confirming that the C4 epimerase was critical for CPSA oligosaccharide production in E. coli (Park et al., 2006).

### Example 3

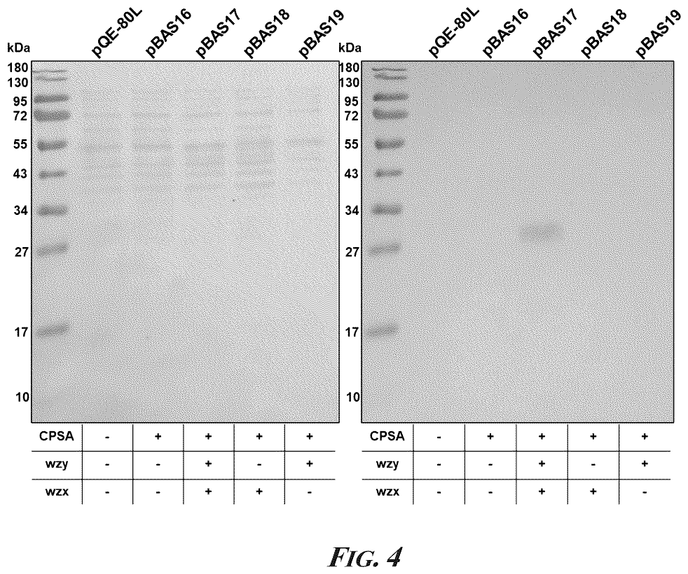

Co-expression of wzx and wzy from B. fragilis Produces CPSA Polymers in E. coli Once we confirmed production of the CPSA repeat unit by our recombinant E. coli strain, we evaluated whether an E. coli flippase or polymerase could catalyze polymer formation or whether the CPSA specific flippase or polymerase from B. fragilis was required. To determine if both Wzx and Wzy were necessary for CPSA polymerization, we constructed plasmids containing only B. fragilis flippase wzx (pBAS18) or polymerase wzy (pBAS19), and B. fragilis wzx and wzy together (pBAS17), along with all other genes required for oligosaccharide biosynthesis (Table 2A). To determine whether CPSA polymers were formed, we evaluated cell lysates using SDS-PAGE and Western blotting with an anti-CPSA antibody serum (FIG. 4). See also Comstock et al., 1999.

TABLE 5
Plasmids used to Determine if Co-expression of wzx_{Bf} and wzy_{Bf} is
Needed for Recombinant Production of CPSA Polymers in E. coli

| Plasmid designation | Genes included |
|---|---|
| pQE-80L (Empty Vector) | [none] |
| pBAS16 | pglF_{Cj}, wbpP_{Vv} wcfRSQOPMN |
| pBAS17 | pglF_{Cj}, wbpP_{Vv} wcfRSQOPMN,wzx, wzy |
| pBAS18 | pglF_{Cj}, wbpP_{Vv} wcfRSQOPMN, wzx |
| pBAS19 | pglF_{Cj}, wbpP_{Vv} wcfRSQOPMN, wzy |

Polymerized CPSA with a molecular weight around 30 kDa was only observed in cells expressing pBAS17 (+wzx_{Bf}, +wzy_{Bf}), and not in cells expressing pBAS16 (−wzx_{Bf}, −wzy_{Bf}), pBAS18 (+wzx_{Bf}), or pBAS19 (+wzy_{Bf}). See FIG. 3. We conclude that CPSA is polymerized in E. coli only when wzx_{Bf} and wzy_{Bf} are co-expressed, or that polymers produced in other strains were below the detection limits of this assay.

### Example 4

### CPSA Oligo- and Polymers are Likely Ligated to Lipid A Core

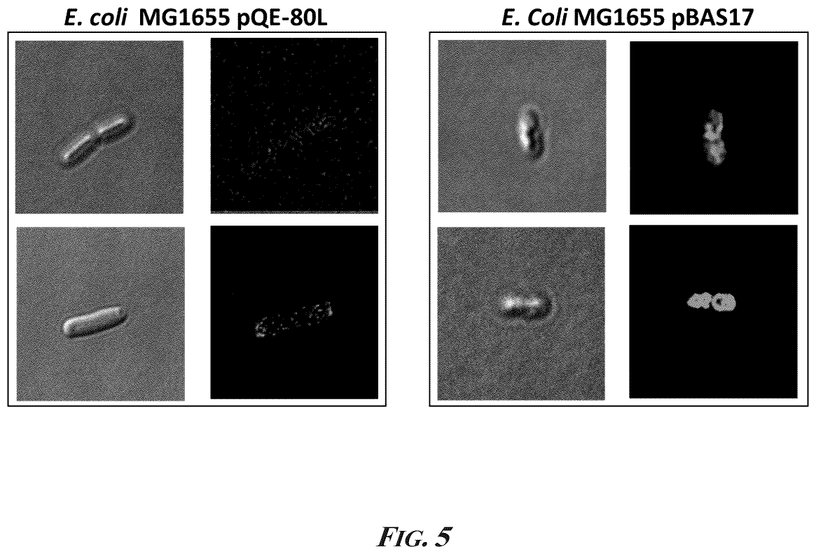

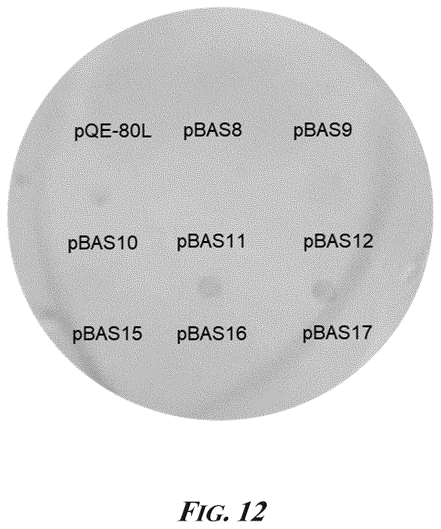

We next sought to determine if CPSA could be detected on the surface of E. coli expressing pBAS17 (+wzx_{Bf}, +wzy_{Bf}). See Table 4. For this and remaining experiments, we transitioned to E. coli MG1655 as the host due to the availability of a small library of relevant mutants. Expression of CPSA in E. coli MG1655 did not qualitatively differ from CPSA expression in DH5α (FIG. 12). Using confocal microscopy, we confirmed that intact cells expressing pBAS17 reacted with anti-CPSA antiserum and FITC conjugated secondary antibodies, while cells expressing an empty plasmid did not (FIG. 5). Using a whole-cell dot blot, we confirmed that pBAS8-15 did not react with anti-CPSA antiserum either but were surprised to find that cells expressing pBAS16 did (FIG. 12, Table 4).

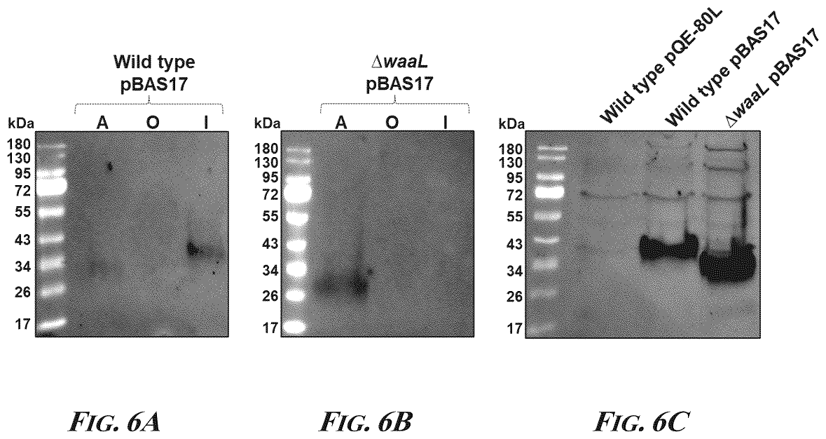

To better understand CPSA glycoforms present in wild-type MG1655, cell lysates were partitioned into organic, aqueous, and insoluble fractions using the Bligh and Dyer method of lipid extraction (Bligh & Dyer, 1959; Henderson et al., 2013). For cells harboring pBAS17, the aqueous fraction was expected to contain water-soluble polysaccharides, potentially including lipid-linked CPSA intermediates (Reid et al., 2021). The organic fraction, typically used for phospholipid isolation, might also contain early BPP-linked CPSA intermediates (Reid et al., 2021). The insoluble phase, largely consisting of LPS would contain LPS-CPSA conjugates (Henderson et al., 2013). Each of these fractions were analyzed by SDS-PAGE and western blotting (FIG. 6). Intriguingly, polymerized CPSA was observed in both the aqueous and insoluble cell fractions in wild-type MG1655 pBAS17, with lower molecular weight polymers observed in the aqueous fraction (˜30 kDa) than the organic fraction (˜40 kDa). Combined with the previous results of surface expressed CPSA (FIG. 5; see also FIG. 12), this strongly suggested that CPSA can be ligated to LPS.

In E. coli, it has been shown that oligo-and polysaccharides other than O-antigens (O-Ag) can be ligated to the lipid A core of LPS, including colanic acid, enterobacterial common antigen (ECA), and peptidoglycan repeat units (Marolda et al., 2006; Meredith et al., 2007; Maciejewska et al., 2020). Additionally, recent developments in recombinant bacterial glycan production demonstrate that non-native glycans may be ligated to the lipid A core (Nothaft et al., 2016; Kay et al., 2022). These glycan modifications of LPS are primarily attributed to the promiscuity of the E. coli O-antigen ligase, WaaL, which appends O-antigen to the heptose residue of the lipid A core (Abeyrathne et al., 2005; Ruan et al., 2012). To evaluate whether WaaL contributes to the production of higher molecular weight CPSA in the insoluble fraction of cell lysates, pBAS17 was overexpressed in an MG1655 ΔwaaL mutant. Analysis of MG1655 ΔwaaL pBAS17 cell lysate fractions revealed CPSA only in the aqueous fraction of cell lysates (FIG. 6) indicating that a ΔwaaL mutant is needed to procure solely aqueous CPSA polymer.

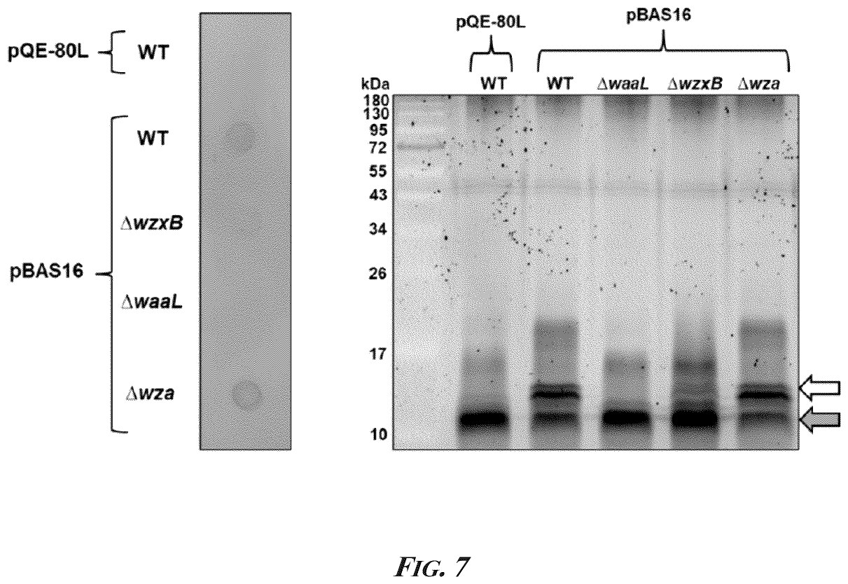

In consideration of these previous findings, we thought it possible that CPSA oligomers and polymers may be ligated to the lipid A core of E. coli cells producing the CPSA repeat unit (pBAS16; −wzx_{Bf}, −wzy_{Bf}) or polymer (pBAS17; +wzx, +wzy_{Bf}), respectively. As demonstrated by a whole-cell dot blot, CPSA surface expression appears to be disrupted in waal mutant expressing pBAS16 (FIG. 7). Based on this finding, we proposed that CPSA may be ligated in oligomeric form to LPS and requires WaaL for ligation to the lipid A core. To confirm this, LPS profiles of pronase treated cell lysates were evaluated using SDS-PAGE and the PRO-Q™ Emerald 300 Lipopolysaccharide Gel Stain Kit stain (Pierce). In MG1655, O-antigen production is disrupted by an IS5 insertion into the wbbL region of the O-antigen operon (Liu & Reeves, 1994). Therefore, LPS profiles of this strain would contain only lipid A core. Fluorescence imaging of PRO-Q™ Emerald 300 stained gels revealed an additional band of a higher molecular weight than lipid A core in pronase treated lysates of cells expressing pBAS16, but not in a waal mutant or those expressing a plasmid control (FIG. 7). We were unable to detect these species with anti-PSA antiserum, which may be due levels below our detection limit. We also tested whether inactivation of wzxB, the O-antigen flippase in E. coli disrupted surface expression of CPSA or altered LPS profiles in cells expressing pBAS16. Western blot signals for ΔwzxB MG1655 pBAS16 were attenuated compared to wild type cells, but still present, indicating that WzxB may, in part, have a role in CPSA oligomer transport and surface expression (FIG. 7). Deletion of E. coli polysaccharide export protein, wza, had no effect on surface expression or LPS profiles of cells expressing pBAS16 compared to wild type.

### Example 5

### Evaluation of Potential Mechanisms of CPSA Polymer Export

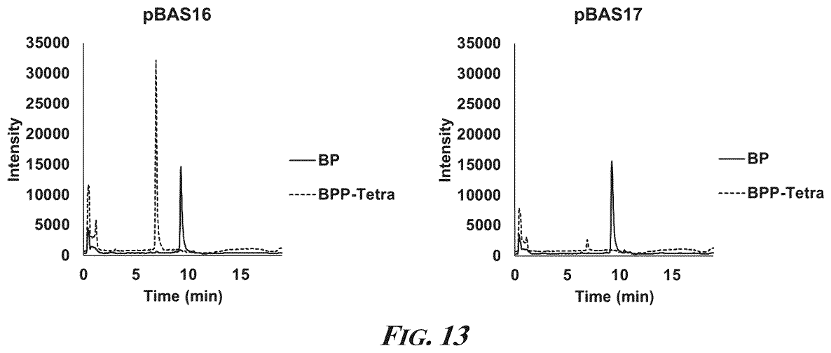

Polysaccharide assembly and export typically occurs via a Wzx/Wzy dependent pathway and its cognate Wza transporter, or via an ATP-binding cassette (ABC) transporter such as observed with LPS transport (Whitfield, 2006). Since our evidence suggests that CPSA may be ligated to LPS and that B. fragilis Wza was not included in our constructs, we considered both of these pathways in E. coli as possible means of CPSA polymer export. We also considered that CPSA oligomers are still present in cells expressing pBAS17 (FIG. 13), even though CPSA polymers are produced. Like cells expressing pBAS16, if CPSA polymers or oligomers are ligated to Lipid A core and transported across the membrane via the well-defined Lpt transport pathway, a waal mutant would abolish CPSA surface expression. Conversely, if CPSA expression was dependent on Wza_{Ec} for export across the outer membrane in cells expressing pBAS17, a wza_{Ec} mutant would no longer exhibit surface expression of CPSA.

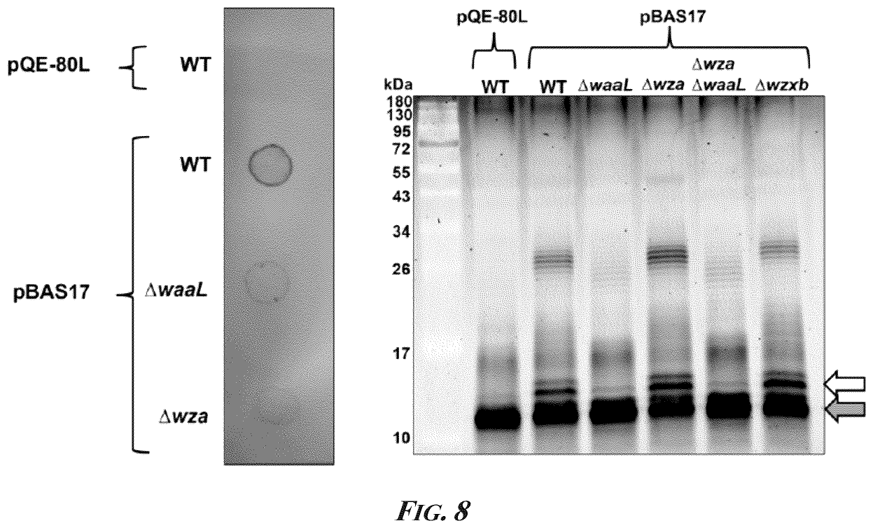

To test whether CPSA polymer export is dependent on E. coli Wza and/or WaaL, we transformed pBAS17 into MG1655 lacking either wza or waal and evaluated CPSA surface expression using a whole-cell dot blot. Surprisingly, a positive, but not as robust signal for CPSA was observed in a Δwza and a ΔwaaL mutant, indicating that neither may be essential for CPSA export (FIG. 8). To assess whether CPSA polymers are appended to Lipid A core in MG1655 pBAS17, we performed an analysis of LPS profiles (FIG. 8). SDS-PAGE of pronase treated lysates and staining with PRO-Q™ Emerald 300 revealed the presence of glycoforms of much higher molecular weights (˜30 kDa), like those observed with anti-CPSA western blotting, and a ladder-like banding pattern in MG1655 pBAS17 (FIGS. 4 and 6). Polymers of slightly lower average molecular weight were observed in the waal mutant when compared to the wza mutant or wild type, suggesting that the polymer is no longer associated with lipid A core when waal is inactivated (FIG. 8). Consistent with our findings in cells expressing pBAS16 (−wzx, −wzy), a low molecular weight band consistent with an oligomer of CPSA appended to lipid A core is no longer apparent in the waal mutant.

### Discussion of the EXAMPLES

A thorough understanding of recombinant polysaccharide biosynthesis in E. coli and hindrances in production at early stages could bolster production and isolation of rare polysaccharides. Our method demonstrates a systematic approach for evaluating individual steps within recombinant Wzx/Wzy dependent polysaccharide systems (Wacker et al., 2002; Ma & Wang, 2019). The efficacy of this method was demonstrated by production of complete polysaccharide of biologically relevant length, and used as a model to address missing or poorly expressed requisites, such as the UDP-GlcNAc C4 epimerase and 4,6-dehydratase not present in the CPSA operon (Kreisman et al., 2007). Additionally, we show that we were able to overcome poorly expressed components (B. Fragilis UngD2) in E. coli by utilizing a well-expressed homolog (Coyne et al., 2008; Mostafavi & Troutman, 2013). Identification of bottlenecks in recombinant systems such as these will provide targeted opportunities to increase polymer quantity.

Moreover, our efforts provided the opportunity to evaluate whether co-expression of wzx_{Bf} and wzy_{Bf} was necessary for the translocation and concomitant polymerization of the CPSA unit oligosaccharide in E. coli. We were surprised to find CPSA polymers could not be detected without the expression of both wzx_{Bf} and wzy_{Bf} (Merino et al., 2016). This supports a previously proposed model that Wzx, Wzy, and Wza form a multi-protein complex wherein the translocase flips the unit oligosaccharide into the periplasm where it is then immediately “passed off” to the cognate polymerase and transporter or ligase (e.g., WaaL; see Feldman et al., 1999; Marolda et al., 2006). Countering this idea, was the expression and purification of a Wzy from E. coli without its associated translocase, and the reconstitution of bacterial polysaccharide biosynthesis in vitro (Woodward et al., 2010). It would be interesting to determine whether Wzx_{Bf} and Wzy_{Bf} colocalize and function as a unit in E. coli.

In the same regard, we found that deletion of wzxB resulted in a significant attenuation of surface expressed CPSA in cells expressing pBAS16 (−wzx, −wzy), with a simultaneous decrease in a signal in SDS-PAGE gels that we propose is lipid A core-linked CPSA oligomer (FIG. 7). In future studies, it would be interesting to see if introduction of wzx_{Bf} in this strain (pBAS19, +wzx_{Bf} only) reverses this effect. The fact that a ΔwzxB mutant did not appear to affect LPS/CPSA profiles in size distribution or signal intensity in cells expressing pBAS17 (+wzx_{Bf,}+wzy_{Bf}) suggests both Wzx_{Bf} functionality in E. coli and its ability to compensate for the lack of wzxB in the WaaL-CPSA pathway (FIG. 8). This also implies promiscuous specificity of E. coli WzxB since it can accept CPSA oligomers, which could prove useful in the design of complex polysaccharide production systems.

Noting the potential activity of both flippases, WzxB_{Ec} and Wzx_{Bf}, we were surprised to find that BPP-tetrasaccharide could still be detected in cells expressing pBAS17, albeit at a much lower intensity (+wzx, +wzy; FIG. 5). This points toward a potential inefficiency in the activity of Wzy_{Bf} polymerase in E. coli and will be a future point of focus for improving CPSA output. Perhaps, this is due to a lack of the B. fragilis CPS transporter proteins in our construct, Wza, Wzz, and Wzc, which are suspected to form an active transporting complex (reviewed by Cuthbertson et al., 2009). Interplay of translocase and chain length regulator (Marolda et al., 2006).

Irregular cell shapes were also noted in strains expressing CPSA plasmids (FIG. 5), as well as anomalous growth curves upon plasmid induction. Future work should be dedicated to incorporating assembled genes into the E. coli genome as a recombinant operon for stability. This could also include the use of various genotypes to determine which gene products in E. coli hinder production and isolation of recombinant polysaccharides, specifically those that utilize the same nucleotide sugars and lipid precursor, BP. We show here, that a ΔwaaL mutant is likely required to procure aqueous, non-lipid A associated CPSA, and has been demonstrated elsewhere in recombinant polysaccharide production systems (Kay et al., 2016). Current studies in our laboratory have implemented a ΔwaaL strain for CPSA isolation and quantification of CPSA production in E. coli. Inclusion of the native CPSA chain length regulator, Wzz which is not included in the B. fragilis CPSA operon, to increase the length and yield of CPSA should also be considered for future studies. Systematic evaluation of the use of different transcription promoters to enhance polysaccharide quantity, recently demonstrated by Kay et al., 2016 will also be considered (Kay et al., 2022).

As such, disclosed herein are recombinant systems for producing polysaccharides including but not limited to CPSA production in bacteria including, but not limited to E. coli, by implementing a stepwise method and strategic incorporation of unrelated, robust glycan modification enzymes. In doing so, the production of each lipid-linked intermediate was confirmed along with CPSA polymers of biologically relevant length. Ultimately, provided herein are effective approaches to evaluating recombinant polysaccharide production, which may be applied to other glycan biosynthesis systems that are not confined within a single operon. These results could aid in the identification of potential targets to increase recombinant polymer quantity and increase the availability of biologically relevant glycans.

Summarily, disclosed herein are recombinant systems for production of CPSA and other polysaccharides in E. coli by implementing a stepwise method and strategic incorporation of unrelated, robust glycan modification enzymes. In doing so, it was confirmed the production of each lipid-linked intermediate and finally, CPSA polymers of biologically relevant length. Ultimately, disclosed herein is an effective approach to evaluate recombinant polysaccharide production, which can be applied to other glycan biosynthesis systems that are not confined within a single operon. These results could aid in the identification of potential targets to increase recombinant polymer quantity and increase the availability of biologically relevant glycans.

### REFERENCES

All references cited in the instant disclosure (indicated by bracketed numbers) as well as those listed below, including but not limited to all patents, patent applications and publications thereof, scientific journal articles, and database entries (such as but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.

Abeyrathne et al. (2005) Functional characterization of WaaL, a ligase associated with linking O-antigen polysaccharide to the core of Pseudomonas aeruginosa lipopolysaccharide. Journal of Bacteriology 187(9):3002-3012.

Baumann et al. (1992) Structural elucidation of two capsular polysaccharides from one strain of Bacteroides fragilis using high-resolution NMR spectroscopy. Biochemistry 31(16):4081-4089.

Bligh & Dyer (1959) A rapid method of total lipid extraction and purification. Canadian Journal of Biochemistry and Physiology 37(8):911-917.

Brubaker et al. (1999) Mitogenic Activity of Purified Capsular Polysaccharide A from Bacteroides fragilis: Differential Stimulatory Effect on Mouse and Rat Lymphocytes In Vitro. The Journal of Immunology 162(4):2235-2242.

Choi et al. (2002) A Unique Structural Pattern Shared by T-Cell-Activating and Abscess-Regulating Zwitterionic Polysaccharides. Biochemistry 41(51):15144-15151.

Cobb et al. (2004) Polysaccharide Processing and Presentation by the MHCII Pathway. Cell 117(5):677-687.

Comstock et al. (1999) Analysis of a capsular polysaccharide biosynthesis locus of Bacteroides fragilis . Infection and Immunity 67(7):3525-3532.

Coyne et al. (2008) Role of glycan synthesis in colonization of the mammalian gut by the bacterial symbiont Bacteroides fragilis . Proceedings of the National Academy of Sciences USA 105(35):13099-13104.

Cunneen et al. (2013) Biosynthesis of UDP-GlcNAc, UndPP-GlcNAc and UDP-GlcNAcA Involves Three Easily Distinguished 4-Epimerase Enzymes, Gne, Gnu and GnaB. PLOS One 8(6):e67646.

Cuthbertson et al. (2009) Pivotal roles of the outer membrane polysaccharide export and polysaccharide copolymerase protein families in export of extracellular polysaccharides in gram-negative bacteria. Microbiology and Molecular Biology Reviews 73(1):155-177.

Datsenko & Wanner (2000) One-step inactivation of chromosomal genes in Escherichia coli K-12 using PCR products. Proceedings of the National Academy of Sciences USA 97:(12):6640-6645.

De Silva et al. (2009) Immunological Response from an Entirely Carbohydrate Antigen: Design of Synthetic Vaccines Based on Tn-PS A1 Conjugates. Journal of the American Chemical Society 131(28):9622-9623.

Feldman et al. (1999) The activity of a putative polyisoprenol-linked sugar translocase (Wzx) involved in Escherichia coli O antigen assembly is independent of the chemical structure of the O repeat. J Biol Chem 274(49):35129-38.

Feldman et al. (2005) Engineering N-linked protein glycosylation with diverse O antigen lipopolysaccharide structures in Escherichia coli. Proceedings of the National Academy of Sciences USA 102(8):3016-3021.

Henderson et al. (2013) Isolation and chemical characterization of lipid A from gram-negative bacteria. Journal of Visualized Experiments (79):e50623.

Kalka-Moll et al. (2002) Zwitterionic Polysaccharides Stimulate T Cells by MHC Class II-Dependent Interactions1. The Journal of Immunology 169(11):6149-6153.

Kay et al. (2016) Recombinant expression of Streptococcus pneumoniae capsular polysaccharides in Escherichia coli. Open Biology 6(4):150243-150243.

Kay et al. (2022) Engineering a suite of E. coli strains for enhanced expression of bacterial polysaccharides and glycoconjugate vaccines. Microb Cell Fact 21(1) 66.

Kreisman et al. (2007) Structure and function relations with a T-cell-activating polysaccharide antigen using circular dichroism. Glycobiology 17(1):46-55.

Liu & Reeves (1994) Escherichia coli K12 regains its O antigen. Microbiology 140(1):49-57.

Ma & Wang (2019) RecET Direct Cloning of Polysaccharide Gene Cluster from Gram-Negative Bacteria. In Bacterial Polysaccharides: Methods and Protocols (Brockhausen (ed)). Springer Science+Business Media, New York, New York. pages 15-23.

Maciejewska et al. (2020) Lipopolysaccharide-Linked Enterobacterial Common Antigen (ECA (LPS)) Occurs in Rough Strains of Escherichia coli R1, R2, and R4. International Journal of Molecular Sciences 21(17):6038.

Marolda et al. (2006) Interplay of the Wzx translocase and the corresponding polymerase and chain length regulator proteins in the translocation and periplasmic assembly of lipopolysaccharide o antigen. Journal of Bacteriology 188(14):5124-5135.

Meredith et al. (2007) Modification of Lipopolysaccharide with Colanic Acid (M-antigen) Repeats in Escherichia coli. Journal of Biological Chemistry 282(11):7790-7798.

Merino et al. (2016) The first sugar of the repeat units is essential for the Wzy polymerase activity and elongation of the O-antigen lipopolysaccharide. Future Microbiology 11(7):903-918.

Mostafavi & Troutman (2013) Biosynthetic Assembly of the Bacteroides fragilis Capsular Polysaccharide A Precursor Bactoprenyl Diphosphate-Linked Acetamido-4-amino-6-deoxygalactopyranose. Biochemistry 52(11):1939-1949.

Nothaft et al. (2016) Engineering the Campylobacter jejuni N-glycan to create an effective chicken vaccine. Scientific Reports 6(1):26511.

Ochoa-Reparaz et al. (2010a) A polysaccharide from the human commensal Bacteroides fragilis protects against CNS demyelinating disease. Mucosal Immunol 3(5):487-495.

Ochoa-Repáraz et al. (2010b) Central nervous system demyelinating disease protection by the human commensal Bacteroides fragilis depends on polysaccharide A expression. J Immunol 2010, 185(7):4101-4108.

Olivier et al. (2006) In vitro biosynthesis of UDP-N,N′-diacetylbacillosamine by enzymes of the Campylobacter jejuni general protein glycosylation system. Biochemistry 45(45):13659-69.

Park et al. (2006) Identification of the Vibrio vulnificus wbpP gene and evaluation of its role in virulence. Infection and Immunity 74(1) 721-728.

Ramakrishna et al. (2019) Bacteroides fragilis polysaccharide A induces IL-10 secreting B and T cells that prevent viral encephalitis. Nature Communications 10(1):2153.

Ranjit & Young (2016) Colanic Acid Intermediates Prevent De Novo Shape Recovery of Escherichia coli Spheroplasts, Calling into Question Biological Roles Previously Attributed to Colanic Acid. J Bacteriol 198(8):1230-1240.

Reid et al. (2021) Tracking Colanic Acid Repeat Unit Formation from Stepwise Biosynthesis Inactivation in Escherichia coli. Biochemistry 60(27):2221-2230.

Reid et al. (2023) Chemoenzymatic Preparation of a Campylobacter jejuni Lipid-Linked Heptasaccharide on an Azide-Linked Polyisoprenoid. ACS Omega 8(17):15790-15798.

Riegert et al. (2017) Structural and Biochemical Investigation of PglF from Campylobacter jejuni Reveals a New Mechanism for a Member of the Short Chain Dehydrogenase/Reductase Superfamily. Biochemistry 56(45):6030-6040.

Ruan et al. (2012) The WaaL O-antigen lipopolysaccharide ligase has features in common with metal ion-independent inverting glycosyltransferases. Glycobiology 22(2):288-299.

Schoenhofen et al. (2006) Functional characterization of dehydratase/aminotransferase pairs from Helicobacter and Campylobacter: enzymes distinguishing the pseudaminic acid and bacillosamine biosynthetic pathways. Journal of Biological Chemistry 281(2):723-732.

Sharma et al. (2017) Complete Tetrasaccharide Repeat Unit Biosynthesis of the Immunomodulatory Bacteroides fragilis Capsular Polysaccharide A. ACS Chem Biol 12(1):92-101.

Tzianabos et al. (2000) T Cells Activated by Zwitterionic Molecules Prevent Abscesses Induced by Pathogenic Bacteria. Journal of Biological Chemistry 275(10):6733-6740.

Tzianabos et al. (2001) Structural rationale for the modulation of abscess formation by Staphylococcus aureus capsular polysaccharides. Proceedings of the National Academy of Sciences USA 98(16):9365-9370.

Wacker et al. (2002) N-linked glycosylation in Campylobacter jejuni and its functional transfer into E. coli. Science 298(5599):1790-1793.

Wang et al. (2000) Structural basis of the abscess-modulating polysaccharide A2 from Bacteroides fragilis. Proceedings of the National Academy of Sciences USA 97(25):13478.

Whitfield (2006) Biosynthesis and assembly of capsular polysaccharides in Escherichia coli. Annu Rev Biochem 75:39-68.

Woodward et al. (2010) In vitro bacterial polysaccharide biosynthesis: defining the functions of Wzy and Wzz. Nature Chemical Biology 6(6):418-423.

Zheng et al. (2020) Capsular Polysaccharide From Bacteroides fragilis Protects Against Ulcerative Colitis in an Undegraded Form. Frontiers in Pharmacology 11:570476.

Zhou et al. (2022) Antigen presenting cell response to polysaccharide A is characterized by the generation of anti-inflammatory macrophages. Glycobiology 32(2):136-147.

It will be understood that various details of the presently disclosed subject matter can be changed without departing from the scope of the presently disclosed subject matter. Furthermore, the foregoing description is for the purpose of illustration only, and not for the purpose of limitation.

1. A method for producing a polysaccharide in a bacterium, the method comprising expressing in the bacterium a plurality of coding sequences, the plurality of coding sequences comprising nucleotide sequences that encode a pglF dehydrogenase gene product, a wbpP UDP-N-acetyl-d-glucosamine C4 epimerase gene product, a wcfR aminotransferase gene product, and a wcfS phospho-glycosyltransferase gene product, and optionally further comprise nucleotide sequences encoding one or more of a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, and further wherein at least one of the coding sequences is heterologous to the bacterium.

2. The method of claim 1, wherein the polysaccharide is a capsular polysaccharide A (CPSA) and the bacterium expresses each of the PglF gene product, the wbpP gene product, the WcfR gene product, the WcfS gene product, the WcfQ gene product, the WcfO gene product, the WcfP gene product, the WcfM gene product, the WcfN gene product, the wzz gene product, the wzx fippase gene product, and the wzy polymerase gene product, at least one of which is heterologous to the bacterium.

3. The method of claim 1, wherein the bacterium is an Escherichia coli (E. coli) bacterium, optionally wherein the E. coli bacterium has reduced or absent waal biological activity.

4. The method of claim 1, wherein:
the pglF gene product is a Campylobacter jejuni gene product; and/or
the wbpP gene product is a Vibrio vulnificus gene product; and/or
the wcfR gene product is a Bacteroides fragilis gene product; and/or
the wcfS gene product is a Bacteroides fragilis gene product; and/or
the wcfQ gene product is a Bacteroides fragilis gene product; and/or
the wcfO gene product is a Bacteroides fragilis gene product; and/or
the wcfP gene product is a Bacteroides fragilis gene product; and/or
the wcfM gene product is a Bacteroides fragilis gene product; and/or
the wcfN gene product is a Bacteroides fragilis gene product; and/or
the wza gene product is an Escherichia coli gene product; and/or
the wzx fippase gene product is a Bacteroides fragilis gene product; and/or
the wzy polymerase gene product is a Bacteroides fragilis gene product; and/or
the wzz gene product is a Bacteroides fragilis gene product.

5. The method of claim 1, wherein the polysaccharide is present in a lipid bilayer of the bacterium, optionally wherein the lipid bilayer is part of the bacterium's cell wall.

6. The method of claim 5, wherein the polysaccharide is a CPSA, optionally an aggregate of two or more CPSA tetrasaccharide monomers.

7. The method of claim 1, wherein the plurality of coding sequences are transcribed to produce a polycistronic messenger RNA (mRNA) molecule in the bacterium.

8. The method of claim 7, wherein the polycistronic mRNA molecule comprises a ribosome-binding site (RBS) operatively linked to a translation initiation codon of each of at least two of the plurality of coding sequences, optionally wherein the polycistronic mRNA molecule comprises a plurality of ribosome-binding sites (RBSs), with an RBS present 5′ to each of at least two, optionally 5′ to each, translation initiation codon present in the polycistronic mRNA, wherein at least one RBS is located 3′ to a translation termination codon of a first coding sequence and 5′ to a translation initiation codon of a second coding sequence present on the polycistronic mRNA molecule.

9. The method of claim 1, wherein the bacterium does not express a functional waal O-antigen ligase gene product.

10. A bacterium comprising a plurality of coding sequences, the plurality of coding sequences comprising nucleotide sequences that encode two or more of a pglF dehydrogenase gene product, a wbpP UDP-GlcNAc C4 epimerase gene product, a wcfR aminotransferase gene product, a wcfS phospho-glycosyltransferase gene product, a wcfQ glycosyltransferase gene product, a wcfO pyruvyltransferase gene product, a wcfP glycosyltransferase gene product, a wcfM UDP-galactopyranose mutase gene product, a wcfN glycosyltransferase gene product, a wza polysaccharide export protein gene product, a wzx fippase gene product, a wzy polymerase gene product, and a wzz gene product, wherein at least one of the plurality of coding sequences is heterologous to the bacterium.

11. The bacterium of claim 10, wherein the bacterium is an E. coli bacterium.

12. The bacterium of claim 10, wherein:
the pglF gene product is a Campylobacter jejuni gene product; and/or
the wbpP gene product is a Vibrio vulnificus gene product; and/or
the wcfR gene product is a Bacteroides fragilis gene product; and/or
the wcfS gene product is a Bacteroides fragilis gene product; and/or
the wcfQ gene product is a Bacteroides fragilis gene product; and/or
the wcfO gene product is a Bacteroides fragilis gene product; and/or
the wcfP gene product is a Bacteroides fragilis gene product; and/or
the wcfM gene product is a Bacteroides fragilis gene product; and/or
the wcfN gene product is a Bacteroides fragilis gene product; and/or
the wza gene product is an Escherichia coli gene product; and/or
the wzx flippase gene product is a Bacteroides fragilis gene product; and/or
the wzy polymerase gene product is a Bacteroides fragilis gene product; and/or
the wzz gene product is a Bacteroides fragilis gene product.

13. The bacterium of claim 10, wherein the plurality of coding sequences are transcribed in the bacterium to produce a polycistronic messenger RNA (mRNA) molecule in the bacterium.

14. The bacterium of claim 13, wherein the polycistronic mRNA molecule comprises a ribosome-binding site (RBS) operatively linked to a translation initiation codon of each of at least two of the plurality of coding sequences, optionally wherein the polycistronic mRNA molecule comprises a plurality of ribosome-binding sites (RBSs), with an RBS present 5′ to each of at least two, optionally 5′ to each, translation initiation codon present in the polycistronic mRNA, wherein at least one RBS is located 3′ to a translation termination codon of a first coding sequence and 5′ to a translation initiation codon of a second coding sequence present on the polycistronic mRNA molecule.

15. The bacterium of claim 10, wherein translation of the polycistronic mRNA molecule in the bacterium results in production of a polysaccharide in the bacterium, optionally wherein the polysaccharide is present in a lipid bilayer of the bacterium, further optionally wherein the lipid bilayer is part of the bacterium's cell wall.

16. The bacterium of claim 15, wherein the polysaccharide is a CPSA, optionally an aggregate of two or more CPSA tetrasaccharide monomers.

17. A method for producing an immunogenic composition, the method comprising mixing an antigenic molecule with a capsular polysaccharide A (CPSA), wherein the CPSA is produced by a bacterium of claim 10.

18. An expression cassette comprising a plurality of coding sequences derived from at least two different bacterial species, wherein the plurality of coding sequences are selected from the group consisting of a pglF dehydrogenase gene coding sequence, a wbpP UDP-N-acetyl-d-glucosamine C4 epimerase gene coding sequence, a wcfR aminotransferase gene coding sequence, a wcfS phospho-glycosyltransferase gene coding sequence, a wcfQ glycosyltransferase gene coding sequence, a wcfO pyruvyltransferase gene coding sequence, a wcfP glycosyltransferase gene coding sequence, a wcfM UDP-galactopyranose mutase gene coding sequence, a wcfN glycosyltransferase gene coding sequence, a wza polysaccharide export protein gene coding sequence, a wzx fippase gene coding sequence, a wzy polymerase gene coding sequence, and a wzz gene coding sequence.

19. The expression cassette of claim 18, wherein the expression cassette comprises a combination of coding sequences selected from the group consisting of:
pglF, wcfR, and wcfS;
pglF, wcfR, wcfS, and wcfQ;
pglF, wcfR, wcfS, wcfQ, and wcfO;
pglF, wcfR, wcfS, wcfQ, wcfO, wbpP, and wcfP;
pglF, wcfR, wcfS, wcfQ, wcfO, wbpP, wcfP, wcfM, and wcfN; and
pglF, wcfR, wcfS, wcfQ, wcfO, wbpP, wcfP, wcfM, wcfN, wzx, and wzy.

20. The expression cassette of claim 18, wherein expression cassette is operatively linked to a promoter such that transcription from the promoter results in the production of a polycistronic mRNA that is translatable to produce polypeptides encoded by the coding sequences.

21. The expression cassette of claim 20, wherein the polycistronic mRNA molecule comprises a ribosome-binding site (RBS) operatively linked to a translation initiation codon of one or more of, optionally each of, the plurality of coding sequences, wherein at least one RBS is located 3′ to a translation termination codon of a first coding sequence and 5′ to a translation initiation codon of a second coding sequence, wherein the first coding sequence and the second coding sequence are adjacent to each other on the polycistronic mRNA molecule, the first coding sequence being present 5′ to the second coding sequence.

22. The expression cassette of claim 18, wherein the polycistronic mRNA molecule is operably linked to a promoter that is active in the bacterium, optionally wherein the promoter is an inducible promoter, optionally an isopropyl-β-d-thiogalactopyranoside (IPTG)-inducible promoter, further optionally wherein the promoter is a T5 promoter.
